(19) United States
(12) Patent Application Publication
(10) Pub. No.: US 2025/0270490 A1
(43) Pub. Date: Aug. 28, 2025

(54) SYSTEM FOR INTEGRATION INTO A FRUIT PROCESSING FACILITY

(71) Applicant: CRUSH DYNAMICS INC., VANCOUVER (CA)

(72) Inventors: Gary Edward Strachan, Summerland (CA); William Leslie Broddy, Summerland (CA)

(21) Appl. No.: 18/590,060

(22) Filed: Feb. 28, 2024

Related U.S. Application Data
(63) Continuation of application No. 17/788,797, filed on Jun. 24, 2022, now abandoned.
Application No. 17/788,797 is a continuation of application No. 16/882,317, filed on May 22, 2020, now Pat. No. 11,584,904.
(60) Provisional application No. 62/852,295, filed on May 23, 2019.

(51) Int. Cl. C12M 1/00; C12G 1/022; C12M 1/42; C12R 1/02; C12R 1/645

(52) U.S. Cl. CPC C12M 23/52; C12G 1/0203; C12M 21/00; C12M 35/04; C12R 2001/02; C12R 2001/645

(57) ABSTRACT

The system comprises one or more transportable bioreactors configured with the ability to conduct aerobic fermentation of fruit biomass. This system integrates three functionalities: transportation regulations, fermentation requirements and food safety/quality control requirements. The transportation design features of the system render the bioreactors transportable to a fruit processing facility, wherein they can be filled with fruit biomass. The transportable bioreactor and processes take into account the requirements of the bioreactor functionalities of the system, given the particular deployment specifications of the system tailored for a certain environment and fruit biomass. Moreover, the design of this system conforms to the minimal requirements for the food safety and quality regulations of the jurisdictions in which the ultimate products will be directly and/or indirectly sold (e.g., as a foodstuff, health supplement, and/or a food ingredient). The integration of these three types of functionalities (transport, bioreactor and sanitation) gives rise to a practical solution for addressing the various problems of wasted foodstuffs such as fruit biomass.

### FIELD

This disclosure pertains to the field of converting fruit biomass into useful nutrient-rich products.

### SUMMARY

The present invention provides a system comprising a transportable bioreactor. The bioreactor is configured for transport via road, rail and/or a body of water, compliant with regulatory requirements. The bioreactor is also configured for fermentation, compliant with sanitation regulations. The transportable bioreactor is capable of collecting fruit biomass at a fruit processing facility, and initiating fermentation at a selected time starting after the fruit biomass is collected in the container.

Such a transportable bioreactor comprises a food-grade container configured for fermentation and providing accessibility for filling, emptying and monitoring and cleaning. It also comprises means to exclude external contamination and means to prevent spillage during transport.

Optionally, the system also includes comprising means for introducing a microbial formulation to initiate fermentation of the biomass in the transportable bioreactor at a selected time. The microbial formulation may comprise acetic acid bacteria and/or fungus inoculant. Appropriate bacteria include Acetobacter and/or Gluconobacter. The microbial formulation may comprise one or more strains of yeast that can ferment under aerobic, microaerophilic and/or anaerobic conditions. It may also comprise one or more enzymes.

The transportable bioreactor optionally includes system monitoring means. In the exemplified embodiment, the fruit processing facility is a winery.

The present invention also involves a process for collecting fruit biomass at a fruit processing facility within a transportable aerobic bioreactor. The process comprises the following steps:
1. providing a transportable aerobic bioreactor at a fruit processing facility;
2. transferring fruit biomass into the transportable aerobic bioreactor at the fruit processing facility;
3. inoculating the fruit biomass within the bioreactor with a microbial formulation;
4. aerobically fermenting the inoculated biomass in the bioreactor until the acidity is at or below pH 4.0; and
5. transporting the bioreactor at a selected time after aforesaid step b, from the fruit processing facility to a processing facility for further processing.
   Desirably, an output of the process is a nutrient-rich “pre-product,” optionally starting with biomass from a winery.

The nutrient-rich pre-product may thus comprise mare with berries and lees derived from a winemaking process. In that embodiment of the invention, one performs the further steps of:
1. introducing said fruit biomass comprising marc into a transportable aerobic bioreactor;
2. grinding the hydrated fruit biomass comprising mare in the bioreactor to generate meal;
3. inoculating the meal in the bioreactor with a microbial formulation; and
4. fermenting the inoculated meal in the bioreactor to generate a fermenting meal.
   The aforesaid process may be augmented by, additionally, after step a, hydrating the fruit biomass comprising mare in the bioreactor until berries swell.

Also, the process may be enhanced by additionally introducing lees, for example first-rack leees, into the bioreactor; emulsifying the lees and fermenting meal in the bioreactor to generate a puree; and transporting the bioreactor to a processing facility where the puree is refined to generate a nutrient-rich product.

An environmental advantage of processes of the present invention is that methane emissions that would otherwise be caused by disposal of fruit biomass in landfills are significantly reduced or eliminated.

The puree may be inoculated in the bioreactor and fermented in the bioreactor to generate a fermented puree. Thereafter, the fermented puree may be refined to generate a refined nutrient-rich product. And then the refined nutrient-rich product may be stabilized to generate a stabilized nutrient-rich product, which is then packaged.

An alternative embodiment of the invention provides a method of converting fruit biomass into an aerobically fermented puree. It comprises the steps of:
a. introducing fruit biomass comprising mare into a transportable aerobic bioreactor;
b. hydrating said fruit biomass comprising mare in the bioreactor until berries swell;
c. grinding the hydrated fruit biomass comprising mare in the bioreactor to generate meal;
d. inoculating the meal in the bioreactor with a microbial formulation;
e. fermenting the inoculated meal in the bioreactor to generate a fermenting meal;
f. introducing lees into the bioreactor;
g. emulsifying the lees and fermenting meal in the bioreactor to generate a fermented puree.

Thereafter, the following steps may be performed: transporting the bioreactor to a processing facility; and refining the fermented puree to generate a refined nutrient-rich product. And then, the following steps may be performed stabilizing the refined nutrient-rich product to generate a stabilized nutrient-rich product; and packaging the stabilized nutrient-rich product.

The following products may be derived from processes of the present invention: a fermented puree; a refined nutrient-rich product; a stabilized nutrient-rich product, which may be introduced into commerce in packaged form. An example of a refined nutrient-rich product produced thereby may contain varietal grape skin, varietal grape seed, and winemaking sediment

### BACKGROUND

The subject matter discussed in the background section should not be assumed to be prior art merely because of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

The technical problem to be solved pertains to industries or businesses that generate and/or use, process or sell fruit, wherein fruit waste is produced and oftentimes sent to landfill, compost, farm-animal feed, etc., increasing greenhouse gases and subtracting from the profitability of the business involved.

According to 2018 data from UN's Food and Agricultural Organization (FAO), the global fruit production was about 870 million metric tons in 2018, with China, being the largest contributor, accounting for about 240.8 million metric tons. The Asia Pacific region accounted for 49% of the market and the second largest region accounting for 13% of the global fruit production was Western Europe. Some of the top fruit producers include India, Brazil and The United States.

Significant losses and waste, however, are becoming a serious nutritional, economical, and environmental problem. The (FAO) has estimated that losses and waste in fruits and vegetables are the highest among all types of foods, and may reach up to 60%. Fruit and vegetable losses and waste also indirectly include wasting of critical resources such as land, water, fertilizers, chemicals, energy, and labor. These immense quantities of lost and wasted food commodities also contribute to immense environmental problems as they decompose in landfills and emit harmful greenhouse gases. These wastes are prone to microbial spoilage causing objectionable odors and environmental problems. In general, it is thought that wastes should be processed using thermal (heating, microwave, radiofrequency, infrared heating, and sterilization) or nonthermal (high hydrostatic pressure, ultrasound, pulsed electric fields (PEFs), irradiation, and pulsed light) technologies, which may affect phytochemicals and other nutrients in the wastes.

One example is the winemaking industry, which produces millions of tons of leftovers and residues, constituting an ecological and economical waste management issue for the wineries. The leftovers and residues include organic wastes, inorganic wastes, wastewater, and emission of greenhouse gases (CO2, volatile organic compounds, etc.) Due to growing issues around groundwater and soil contamination, wineries send most of it to the landfill, costing the winery fees for bin drop-off, removal, haulage and tipping fees in addition to winery management costs. Addressing these issues in an appropriate manner places a financial burden on most of the wineries, especially the smaller ones.

The winemaking process generates two major residues, which can be harvested. The major residues from the winemaking process after the de-stemming and crush steps are known as derivatives. Derivatives comprise grape marc (pomace) and lees. For every two bottles of wine made, typically the equivalent of one bottle of derivatives is produced. Winery derivatives comprise:
a) marc (pomace) consisting of grape skin, grape pulp and grape seed derived from varietal grapes, which have been crushed and pressed as part of the winemaking process; and
b) lees consisting of spent wine yeast, tartaric acid, grape skin pigment and grape pulp sediment, which have been extruded from the wine after fermentation and again after aging.

Grape marc provides substantial nutritional potential as supplements and to fortify food. For example, 15 grams (˜1 tbsp.) of powdered derivative may contain up to 900 mg of phenols, 150 mg of tannins (catechin), 2000 mg of protein, 180 mg of potassium, 120 mg of magnesium, 4 mg of iron, 4% DV of riboflavin, 125% DV of vitamin E and 3% DV of vitamin K).

In general, wine lees is residue that forms at the bottom of wine containers consisting of: 1) first and second-fermentation lees, which are formed during the alcoholic and malolactic fermentations, respectively (herein, lees); 2) during storage or after treatments (herein, first-rack lees); and 3) aging wine lees formed during wine aging in wood barrels collected after the filtration or centrifugation of the wine (herein, second-rack lees), The main characteristics of wine lees are acidic pH (between 3 and 6), a chemical oxygen demand above 30,000 mg/L, potassium levels around 2500 mg/L, and phenolic compounds in amounts up to 1000 mg/L Approximately 30% of red wine lees are protein that is produced from yeast cell wall material, which contains 30-60% 3-b-D-glucan in dry weight.

Derivatives are used in livestock and poultry feed to extend the shelf-life of milk, dairy by-nutrient-rich products, and meat. There is extensive research on the anti-microbial benefits as a replacement for antibiotics for poultry and livestock. There is even research showing that it can cut bovine methane emissions by 30%

Although there is an identified market for these derivatives, the current processes used to transform it into shelf-stable nutrient-rich products creates a carbon footprint, is prohibitively expensive and causes significant loss in the quality in the derivatives.

The extraction of useful nutrient-rich products from wine derivatives is known in the art. However, most of these processes seek to isolate a specific compound, require multiple steps, and/or require drying the nutrient-rich product into a powder that can be easily sold in capsule, tablet, powder form, etc. Drying the nutrient-rich product and/or using chemical processes to isolate nutrient-rich products therefrom can diminish the bioavailability of the biomolecules desired in the final nutrient-rich products.

It is widely recognized that the nutrient value of foods has been diminishing since at least the 1950's, such that a need has developed for cost effective strategies to fortifying foods in the food supply, incorporating the resources of a fruit processing facility to make adaptation easily accessible for the business.

There is tremendous value in monetizing these derivatives. The issue today is economics; finding a cost-effective way to process derivatives in an ecological manner, without losing flavour and nutrition.

### Overview of a Derivative-Conversion Process in Winemaking

With reference to FIGS. 1 and 2, this section will introduce an overview for two embodiments of a wine derivative-conversion process. The steps of a traditional general winemaking process are labeled with alpha characters (A, B, C, etc.). The steps within a general derivative-conversion process for generating bioactive nutrient-rich products are labelled with numeric characters (1, 2, 3, etc.).

In Step A 102, the winery either picks or buys varietal wine grapes that are optimized for winemaking and they are transported to the winery “crush pad” to be processed. The grapes may be destemmed or not destemmed prior to Step B 104, depending upon the preferred method of wine production, and loaded into the grape crusher. During Step B 104, the grapes are masticated so that the juice (must) can be separated from the skins, pulp and seeds. If the crushed grapes and must are to be used in a white or rose style wine, they are immediately treated according to the process of Step C 108.

If the crushed grapes and must are to be used in a red or “orange” style wine, they are processed in Step B1 106, which entails loading the crushed grapes and must into the fermentation tank where yeast is added to initiate alcoholic fermentation. When the alcoholic fermentation process has terminated, the wine (“free run wine”) and the crushed grapes are further processed in Step C 109. Typically, the free run wine is pumped off into tanks and the skins subjected to step C 108, where they are pressed to extract the remaining juice and wine. The press wine may optionally be blended with the free run wine at the winemaker's discretion.

Thus, Step C 108 is applied to either the must derived from Step B 104 (e.g. in the production of white wine) or to the alcoholic fermented wine (free run wine) derived from Step B1 106 (e.g., in the production of red wine). During Step C 108, the crushed grapes and either, the unfermented must derived from Step B 104 or the free run wine derived from Step B1 106, are loaded into the press so that the must or free run wine can be squeezed from the mare 109 (solid matter). The must, destined to become a white wine, is loaded into a fermentation tank where yeast may be added to initiate alcoholic fermentation of the must. The press wine produced from Step C 108, which has now been separated from the marc 109, in step D 110 is inoculated with specific strains of bacteria (lactobacter) to initiate malo-lactic fermentation to convert “crisp, green apple” malic acid to “soft, creamy” lactic acid to soften the taste of the wine. The marc 109, which is traditionally treated as food waste, is immediately removed from the “food preparation” area (crush pad).

One example of a derivative-conversion process entails rehydrating the marc 109 with enough water to saturate the marc 109. After the berries have swollen, the saturated marc is ground into fine particulates and is then inoculated with microbials to cause fermentation and dissolution of solid particles. This process can generally last for approximately 2 to 6 weeks before proceeding to Step 2 218, 224.

During Step D 110 of the general winemaking process for white wine, the must is fermented until it turns into wine. As part of this step the spent yeast, potassium acid tartrate, skin and pulp particulates settles to the bottom of the fermentation tank. As mentioned above, the press wine is usually subjected to malo-lactic fermentation during Step D, during traditional red wine making practices.

During Step E 112 of the winemaking process, the settled particulates are separated from the wine by drawing the wine off of the top in a process known as racking and placed into oak, steel or ceramic vessels for aging. This particulate matter, suspended in a residual amount of wine is referred to herein as first-rack lees 113. During traditional winemaking, the first-rack lees 113 is typically treated as food waste and is immediately removed from “food preparation” area.

During the process of derivative-conversion, Step 2 214, 218, 224, incorporates the first-rack lees 113 into the bioreactor 300 where it is emulsified with the biomass, which is then further fermented.

During Step F 114 of winemaking, the wine is aged for 2 to 60 months, depending on the grape varietal and winemaking technique. The wine is then filtered to remove any residual lees, herein referred to as second-rack lees 115, and placed into bottles, kegs or waterproof boxes. During traditional winemaking, the second-rack lees 115 typically is treated as food waste and is immediately removed from the “food preparation” area. During the process of derivative-conversion, however, as illustrated in FIG. 2 the second-rack lees 115 can be transferred to the bioreactor 300 during Step 2A 214, 218 224, 228, 230, the biomass is then further-fermented to generate a further-fermented purée 232.

FIG. 2 illustrates that the first rack-lees 113 from Step E 112 is added to the emulsion created during Step 1 204, 206, 208, 210, mixed and then further fermented during Step 2 218, 224 until the second-rack lees 115 are ready, at which time they are transferred to the bioreactor and fermentation is continued until the further-fermented nutrient-rich product 232 achieves the desired flavour profile, nutrient value, and PH level.

FIGS. 1 and 2 describe that at Step 3 234, the fermented purée 225 or further-fermented purée 232 are refined using filtration, homogenization, other techniques, or a combination of techniques. At this step, excess water is removed along with any undesirable particulates or bi-nutrient-rich products, such as sulfur, bentonite, etc.

FIGS. 1 and 2 teach at Step 4 238 that refined nutrient-rich product 236 is rendered shelf-stable through pasteurization or correction of PH level through further fermentation and thereby converted to stabilized nutrient-rich product 240.

FIGS. 1 and 2 illustrate that during Step 5 242, the stabilized nutrient-rich product 240 is packaged into consumer, culinary and/or industrial vessels. It is then stored for shipment.

The stabilized nutrient-rich product 240 could be in liquid, paste or powder format based on the needs of the end-user.

### BRIEF DESCRIPTION OF THE FIGURES

The foregoing and other features and advantages of the subject matter disclosed herein will be made apparent from the following detailed description taken in conjunction with the accompanying drawings, in which:

FIG. 1 illustrates steps involved in separating marc and lees from a general winemaking process.

FIG. 2 provides steps of separating marc and lees from a general winemaking process, wherein the process includes the addition off second-rack lees.

FIG. 3 illustrates a schematic diagram side-view of a transportable aerobic bioreactor according to one embodiment comprising a vessel lid attached by hinging means within this system.

FIG. 4 illustrates a schematic diagram side-view of a transportable aerobic bioreactor according to one embodiment comprising a vessel lid, an optional airlock and a positive displacement seal.

FIG. 5 shows a schematic diagram side-view of bioreactor vessel and vessel lid for a transportable aerobic bioreactor according to one embodiment of the system.

FIG. 6 is a schematic diagram top-down view of a lid for a transportable aerobic bioreactor according to one embodiment of the system.

FIG. 7 is a schematic diagram of the bottom of an inner chamber of a transportable aerobic bioreactor according to one embodiment of the system.

FIG. 8 is a simplified block diagram of the system comprising a remote network according to one embodiment of the system.

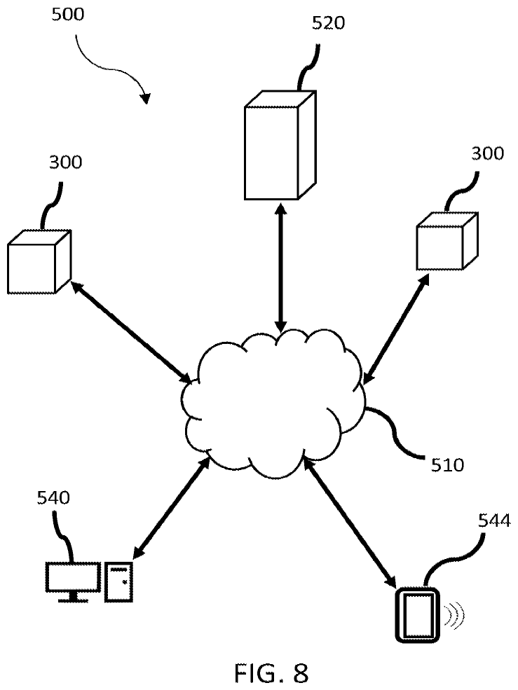

FIG. 9 is a schematic diagram side view for a blow mold for making the inner chamber of a transportable aerobic bioreactor according to one embodiment of the system.

FIG. 10 provides one example of the system, according to one embodiment illustrating the integration of the system with the workflow of a winery.

FIG. 11 is a flow diagram showing one embodiment of the steps of a derivative conversion process, in addition to intermediates and nutrient-rich products generated throughout. As indicated at 226, the description of the process continues in FIG. 12.

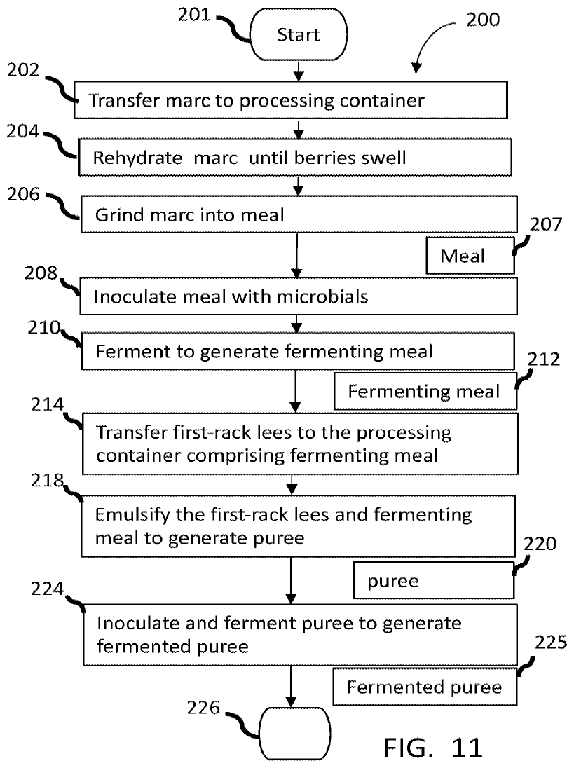

FIG. 12 is a continuation of the flow diagram of FIG. 11, continuing at 226.

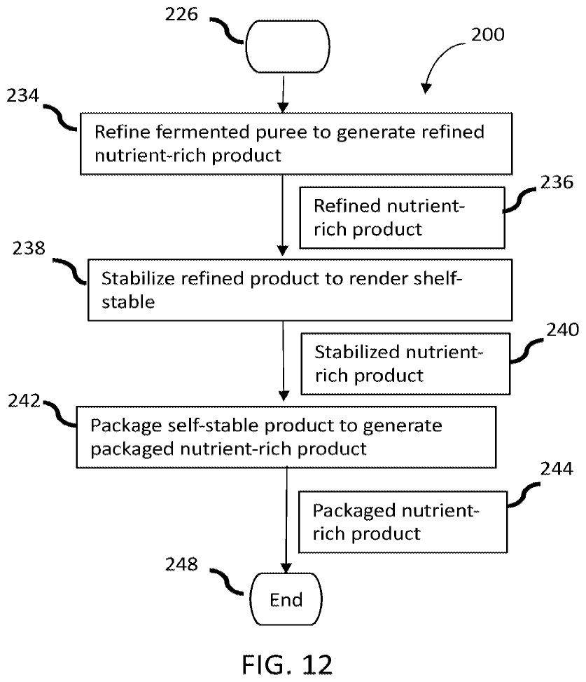

FIG. 13 is a flow diagram showing one embodiment of the steps of a derivative-conversion process in addition to intermediates and nutrient-rich products generated throughout. As indicated at 226, the description of the process continues in FIG. 14.

FIG. 14 is a continuation of the flow diagram of FIG. 13, continuing at 226.

FIG. 15 provides one embodiment of a method of performing the business method described herein.

FIG. 16 is schematic diagram overhead view illustrating one aspect of integrating the system into a fruit processing facility such as a winery, according to one embodiment of the system.

FIG. 17 is a schematic diagram overhead view depicting a service module surrounded by a number of transportable bioreactors, according to one embodiment of the system.

FIG. 18 shows a top angle view of the lid and support structure of a transportable aerobic bioreactor, according to one embodiment of the system.

FIG. 19 illustrates one embodiment of a series of transportable bioreactors located on site at a fruit processing facility, according to one embodiment of the system

FIG. 20 illustrates one embodiment of transportable bioreactors, according to one embodiment of the system.

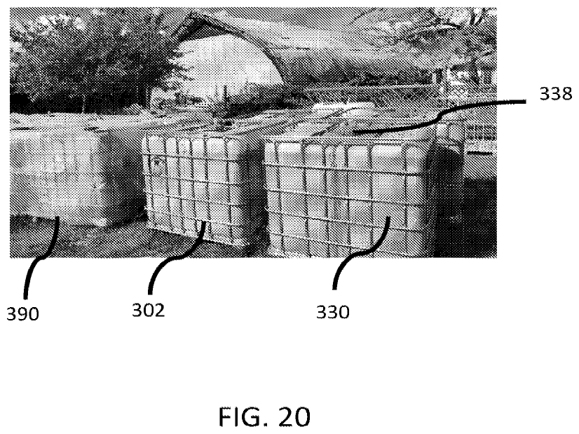

FIG. 21 shows a schematic diagram of a cylindrical bioreactor vessel and vessel lid for a transportable aerobic bioreactor according to one embodiment of the system.

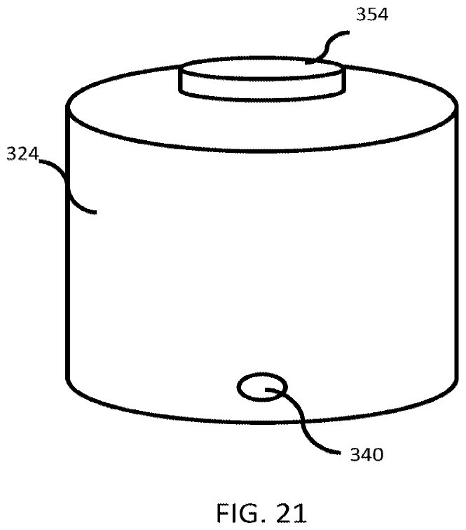

FIG. 22 shows a schematic diagram of a cylindrical bioreactor vessel for a transportable aerobic bioreactor according to one embodiment of the system.

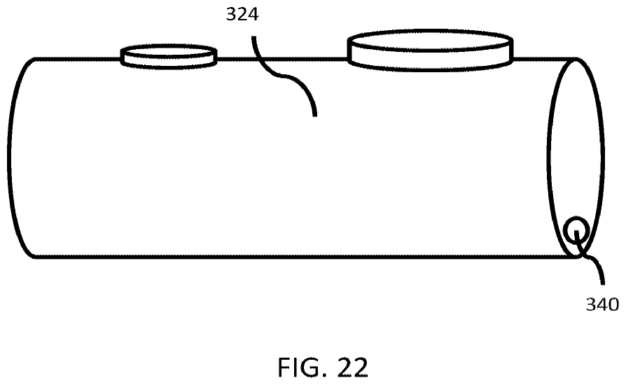

### DETAILED DESCRIPTION

The description set forth below in connection with the appended drawings is intended as a description of various embodiments of the described subject matter and is not necessarily intended to represent the only embodiment(s). In certain instances, the description includes specific details for the purpose of providing an understanding of the described subject matter. However, it will be apparent to those skilled in the art that embodiments may be practiced without these specific details. In some instances, structures and components may be shown in block diagram form in order to avoid obscuring the concepts of the described subject matter. Wherever possible, the same reference numbers will be used throughout the drawings to refer to the same or the like parts.

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning. i.e., a meaning other than that understood by skilled artisans, such a special definition is expressly set forth in the specification in a definitional manner that directly and unequivocally provides the special definition for the term or phrase.

The term comprising means “including but not limited to,” unless expressly specified otherwise. When used in the appended claims, in original and amended form, the term “comprising” is intended to be inclusive or open-ended and does not exclude any additional, unrecited element, method, step or material. The term “consisting of” excludes any element, step or material other than those specified in the claim. As used herein, “up to” includes zero, meaning no amount is added in some embodiments.

The term “about” generally refers to a range of numerical values (e.g., +/−1-3% of the recited value) that one of ordinary skill in the art would consider equivalent to the recited value (e.g., having the same function or result). In some instances, the term “about” includes the values disclosed and may include numerical values that are rounded to the nearest significant figure. Moreover, all numerical ranges herein should be understood to include all integer, whole or fractions, within the range recited.

Any reference in the specification to “one embodiment” or “an embodiment” means that a particular feature, structure, characteristic, operation, or function described in connection with an embodiment is included in at least one embodiment. Thus, any appearance of the phrases “in one embodiment” or “in an embodiment” in the specification is not necessarily referring to the same embodiment. Further, the particular features, structures, characteristics, operations, or functions may be combined in any suitable manner in one or more embodiments, and it is intended that embodiments of the described subject matter can and do cover modifications and variations of the described embodiments.

It must also be noted that, as used in the specification, appended claims and abstract, the singular forms “a,” “an,” and “the” include plural referents unless the context clearly dictates otherwise. That is, unless clearly specified otherwise, as used herein the words “a” and “an” and the like carry the meaning of “one or more” or “at least one.” The phrases “at least one.” “one or more,” “or,” and “and/or” are open-ended expressions that can be both conjunctive and disjunctive in operation. For example, each of the expressions “at least one of A, B and C,” “at least one of A, B, or C,” “one or more of A. B, and C,” “one or more of A, B, or C.” “A, B, and/or C,” and “A, B, or C” can mean A alone, B alone, C alone, A and B together, A and C together, B and C together, or A, B and C together. It is also to be noted that the terms “comprising,” “including,” and “having” can be used interchangeably.

As used herein, the term, fruit biomass is used to denote fruit material, including whole fruit, pressed fruit, pressed grapes from the wine making, and pitted fruit.

As used herein, the term, fruit processing facility is used to denote a location where fruit is processed and some portion thereof tends to be diverted to waste. Examples comprise fruit production businesses, orchards, wineries, farms, fruit packers, etc.

In one embodiment, the system could be used to convert vegetable biomass, for example, sugar beets, carrots, and other vegetables which could provide good candidates for fermentation, etc., to assist a vegetable processing facility to manage its previously-considered waste into nutrient-rich products, which can optionally provide a novel revenue stream for the vegetable processing facility.

Described herein is a system for the conversion of fruit biomass such as fruit waste and winery derivatives into nutrient rich products comprising antioxidants and other bioactive molecules. Integration of this system into a fruit processing facility such as a farm or winery assists the fruit processing facility to manage its previously-considered waste into nutrient-rich products, which can optionally provide a novel revenue stream for the fruit processing facility. These nutrient-rich products can be used as natural flavour, texture and color enhancers, in addition to nutritional ingredients to fortify processed foods and consumer recipes. They can also be used as health supplements.

### The System

The system comprises one or more transportable bioreactors configured with the ability to conduct aerobic fermentation of fruit biomass in an autonomous and/or semi-autonomous manner, requiring minimal oversight. This system integrates three functionalities: transportation regulations, fermentation requirements and food safety/quality control.

The flexibility of the design for each embodiment of the system is such that the details are determined by the specifications of the regulations for transportation, food safety/quality control, fermentation requirements in the local environment and jurisdiction. The design for fermentation and process instruction can be found in one or more books/reference manuals, such as, for example, “Wine Microbiology”. By Fugelsang and Gump, “Food Microbiology.” “Food Fermentations,” “Indigenous Fermented Foods,” or “Food Microbiology: Fundamentals and Frontiers”, edited by Michael P. Doyle, Larry R. Beuchat, and Thomas J. Montville.

In accordance with the present invention, the design of this system conforms to the minimal requirements for the transportation of substances that must be adhered to in accordance with local transportation regulations. These design features of the system render the bioreactors readily transportable (i.e., the transportation functionalities). In accordance with the present invention, the fermentation vessel and processes take into account the requirements of the bioreactor functionalities of the system, given the particular deployment specifications of the system tailored for a certain environment and fruit biomass. The integration of these two types of functionalities (transport and bioreactor) gives rise to a practical solution for addressing the various problems of wasted foodstuffs such as fruit biomass.

Moreover, in accordance with the present invention, the design of this system conforms to the minimal requirements for the food safety and quality regulations of the jurisdictions in which the ultimate products will be directly and/or indirectly sold (e.g., as a foodstuff, health supplement, and/or a food ingredient). For example, the system is designed such that mold and other microbial contamination does not infiltrate the system and cause toxic substances such as mycotoxins and microbial volatile organic compounds (mVOC's) to contaminate the ultimate products of the system. For example, when constructing the system and incorporating various elements therein attention needs to be provided to all aspects, especially those surfaces which are hard to clean, for example, weld joints, which are somewhat rough and sometimes may even have excessive pitting, resulting in a portion of the weld joint which is very hard to clean. Soil and other impurities get trapped in this area and attract microbial growth. This can become a huge problem if not taken care of in the early stage of the microbial growth. Thus, even the welding of the container need follow the standards for the practice known as sanitary welding, as provided for by example, the American Welding Society (AWS).

In general, food processing operations and retailers must comply with the various Hazard Analysis Critical Control Point (HACCP) sanitary standards and regulations promulgated by various state and local Health Departments, Food processing operations are certified by a third-party agency who will list the sampling points and provide safety certification. Examples of certifications comprise Good Manufacturing Practices (GMP), HACCP or the International Standards Organization (ISO).

The design and construction of the transportable bioreactor could follow the recommendations of 3-A Sanitary Standards, Inc. This organization maintains a large inventory of design criteria for equipment and processing systems developed for the so-called sanitary market using a modern consensus process based on ANSI requirements to promote acceptance by USDA, FDA and state regulatory authorities. One example pertains to sanitary rotary positive displacement pump types, which are designed with certain common characteristics to facilitate sanitation. Among these are an ability to rapidly tear down or open the fluid flow pathway of the pump for easy and thorough inspection and cleaning, often without the need for tools; the extensive use of stainless steels to assure non-contaminating and non-corroding liquid pumpage contact surfaces; the use of simple sanitary seal structures; the minimization or elimination of areas within the interior of the pump which could cause contamination of the pumpage; low RPM operation for gentle liquid handling; ability to operate at elevated temperatures; an ability to pump liquids ranging from very low viscosity to very high viscosity; and conformance to generally recognized sanitary standards, particularly the Standards For Centrifugal and Positive Rotary Pumps For Milk and Milk Products, 02-09, as promulgated in the US by the 3-A Sanitary Standards Symbol Administrative Council. This standard applies not only to dairy uses but also is the de facto standard for most sanitary pump uses.

In one embodiment, the system can be transported to a fruit processing facility such as a winery or an orchard, such that the fruit, pomace and/or biomass that would normally be discarded or constitute a lower value stream such as compost or livestock feed, can be placed into the bioreactor on site, and fermentation can be initiated and conducted on location. Alternatively, once filled, the bioreactors can be transported to another location, such as a field, parking lot, warehouse, etc. where fermentation can be initiated and conducted with minimal supervision. When it is determined appropriate, the transportable bioreactors can be transported to a processing facility, wherein the fermented can be further processed, refined and/or stabilized for use as a food additive, health supplement, or other such product.

In one embodiment, each transportable bioreactor optionally comprises a monitoring system comprising a temperature sensor, an acid sensor such as a pH sensor, and optionally a Brix (sugar level) sensor and sensors for various volatile organic compounds (VOC's), optionally an RFID tag for identification, etc, wherein the reaction within the bioreactor vessel can be monitored wired and/or wirelessly, and/or remotely. In one embodiment, each bioreactor does not comprise a monitoring system, but the progress is assessed in some manner.

### A Transportable Bioreactor

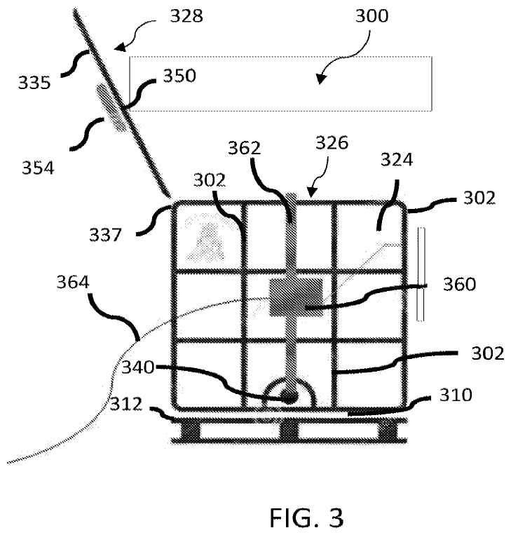

One embodiment of a transportable bioreactor 300 is depicted in FIG. 3 and comprises a bioreactor vessel 320 and an outer support structure 302.

### The Outer Support Structure

The outer support structure 302, is designed to meet the transportation requirements of the jurisdiction in which it is to be deployed. For example, the size of the transportable bioreactor will be tailored to meet the specification of the particular embodiment of the system. Some of the criteria that may be relevant include transportation restrictions. For example, a practical incremental unit limit for highway transportation in North America is 20 tons, because truckers are allowed to load 20 tons on a tandem drive truck, or 40 tons on a semi-trailer or 60 tons on a tandem trailer Many industrial containers are built for 20 tons because machinery is designed with hydraulic systems that can lift 20 tons and place the loaded container on the back of a truck.

There are also height limitations for containers loaded onto the back of a truck, such that the truck plus its contents can pass under bridges, within tunnels, etc. In one embodiment, the transportable bioreactors are rectangular shaped, wherein they can be placed vertically in the field, on location to minimize their footprint and then placed horizontally on a truck so as to meet the height limitations for trucking.

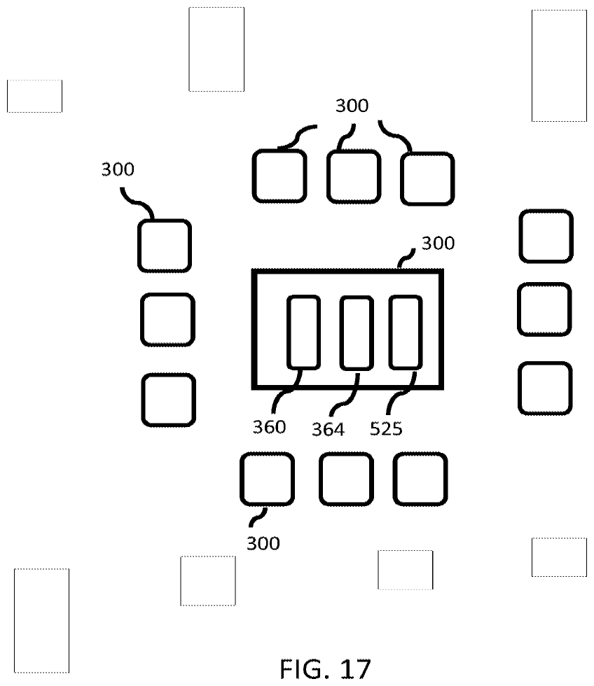

There are regulations pertaining to how containers must be secured if they are to be transported. For example, FIG. 17 depicts a security guard bar 320, which is required in some jurisdictions to ensure that the inner container remains securely fastened within the outer support structure 302, which is also securely fastened to the transportation vehicle. This is especially important if the inner container has a vessel lid 334, that could fall off during transportation and land on a highway causing potentially fatal car accidents.

The outer support structure 302, can be any shape that meets the specifications for a particular use. For example, the outer support structure 302 may be cylindrical, generally cylindrical with squared corners, square, rectangular, etc. or some variation thereof. In general, transportable bulk containers tend to be square or rectangular to maximize space utilization. The transportable bioreactor, however, also is designed to take into account the design requirements of the bioreactor vessel and the food quality and safety requirements, which may dictate different configurations of the outer support structure 302.

The outer support structure 302, may be solid, cage-like, some combination thereof or other design that meets the specifications for a particular application or use. The design specifications may take into account transportation requirements, easy of moving the transportable bioreactor, for example by including a pallet-like structure on the base to facilitate lifting it onto or off of a transportation vehicle in addition to moving it around a fruit processing facility, for example. The design specifications may take into account devices, tubing, protection means, monitoring means, etc. that may be attached to the outer support structure 302. The design specifications may take into account features to facilitate meeting food quality & safety requirements, such as security features and/or ease of inspection features.

With reference to FIGS. 3 & 4, according to one embodiment, which has a largely square and/or rectangular base 310, the outer support structure has four outer “walls” and a base 310, which are all interconnected. The outer walls may be solid, cage-like and/or some combination thereof. The cage-like structure is described in this non-limiting embodiment. The base 310 may additionally incorporate the function of a pallet 312 into the structure of the outer base 310 of the support structure. When the outer support structure 302 is cage-like, there are generally vertical bars and horizontal bars and/or some combination thereof. The outer support structure 302 may be used to attach accessory devices in addition to providing protection to the bioreactor vessel 320, located therein.

### Means to Prevent Spillage During Transport

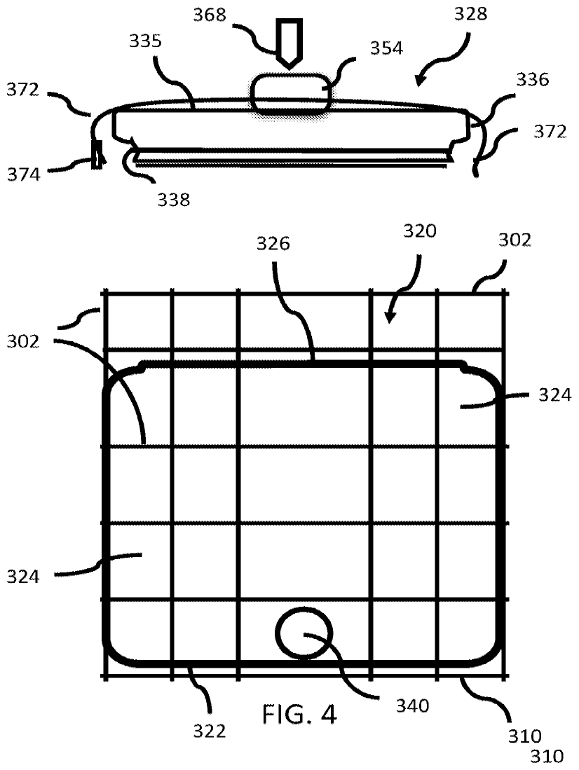

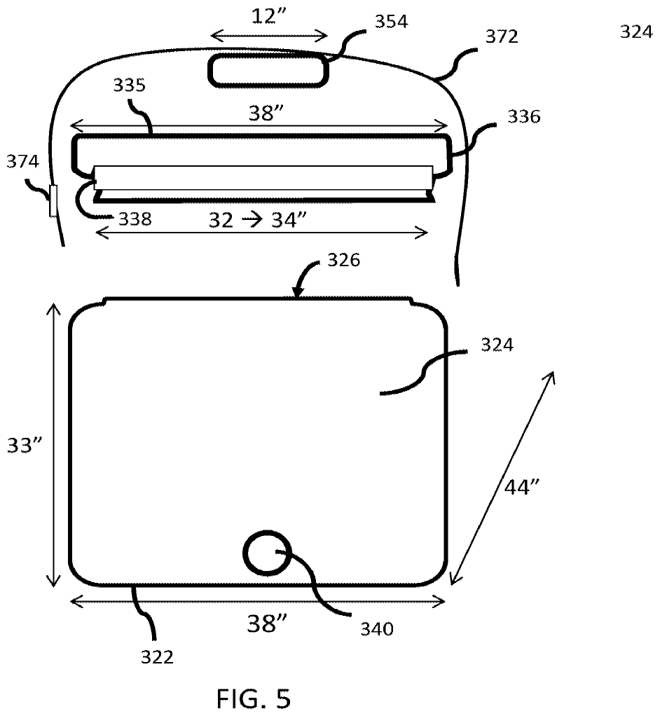

The outer support structure 302 meets the regulations of the transport regulatory bodies in the jurisdictions of interest. For example, most transport regulatory bodies require means to prevent spillage during transport, generally being a security feature to ensure that the lids stay on the transport containers. In some jurisdictions, this is accomplished by securing a bar over the top of the inner container lid, wherein the bar is attached to the side walls. In some jurisdictions, it may be possible to replace the bar with a strap 372, such as depicted in FIGS. 4, 5 & 6, that can be easily attached to the side walls of the outer support structure 302. The strap 372, could comprise a security attachment means 374, such as a lock, zip-tie or other such attachment means 374. This strap could also serve to keep the lid tightly secured to the container to ensure the integrity of one or more positive displacement seals 338, when fruit biomass has been placed inside the container, especially when aeration and fermentation occurs and it is important to maintain a positive pressure within the bioreactor vessel 330 of the transportable bioreactor 300.

### The Bioreactor Vessel

The footprint or the bottom surface of a bioreactor vessel 320, can be square, rectangular, circular, oval or other shape determined to best meet the specifications of the fermentation, tailored for a specific deployment (embodiment) of the system. For example, it may be determined that a cylindrical bioreactor vessel 320 with a bottom surface that is circular or oval shape for the bioreactor vessel 320 may optimize oxygen diffusion and mixing to prevent anaerobic fermentation in locations within the bioreactor vessel such as in the corners of the bioreactor vessel 320 in addition to making cleaning of the vessel much easier than if corners are present.

### A Generally Cubic Bioreactor Vessel

Features of a bioreactor vessel will be introduced with reference to generally cubic bioreactor vessel 302 with a square or rectangular base, but this is not intended to limit the design or description of the system. In one embodiment, a square or rectangular bioreactor vessel 320 has four side walls 324 (only one side is visible in FIGS. 3 & 4), a bottom surface 322, a vessel lid 328, which is optionally attached (see FIG. 3) via hinging means 337 to one wall of the bioreactor vessel 320 of the transportable bioreactor 300. In one embodiment as shown in FIG. 4 a vessel lid 328 is not attached via hinging means. In one embodiment, the lid is not removable from the bioreactor vessel, but sufficiently large access ports are configured in the lid that the lid itself does not need to be removed.

### A Generally Cylindrical Bioreactor Vessel

One embodiment of the bioreactor vessel 320 is cylindrical and/or largely cylindrical. In one embodiment, the transportable bioreactor is modeled on a tank container, optionally built to ISO standards (a.k.a. tanktainer, ISO tank, or an intermodal tank container). For example, this embodiment comprises a bioreactor vessel 320 constructed of stainless steel surrounded by an insulation and protective layer of usually polyurethane and aluminum. The outer support structure 302 comprises a steel frame.

In a cylindrical embodiment, the bioreactor vessel 320 includes a bottom surface 322 and a side wall 324 that extends from the bottom surface 322. In one embodiment, the side wall 324 is substantially cylindrical, but in other embodiments, the vessel may include one or more side walls and have other suitable shapes. The side wall(s) 324 define at least one central opening 326 that is axially opposite the bottom surface 322 and may be configured as an access port, which may be used to access the interior of the bioreactor vessel 320.

### A Vessel Lid

A vessel lid 328 is disposed adjacent the central opening 326. The vessel lid 328 includes a top wall 355 and a side wall 336 that extends axially from the top wall 355. The side wall 336 of the vessel lid 328 engages the side wall 324 of the bioreactor vessel 320. In one embodiment, wherein the bioreactor vessel 320 is circular, and wherein the vessel lid 328 would be screwed onto the bioreactor vessel 320, a radially inward surface of the side wall 324 of the bioreactor vessel 320 defines threads that mate with threads defined on a radially outward surface of the side wall 336 of the vessel lid 328 to couple the vessel lid 328 to the vessel bioreactor vessel 320 adjacent the opening 326. In other embodiments, alternative mechanisms for coupling the vessel lid 328 adjacent to the central opening 326 of the bioreactor vessel 320 may be used, such as a friction fit, snap fit, clamp, etc. The vessel lid 328, according to various embodiments, is made of ceramics, plastics, resins, metal (e.g., steel), other suitable rigid material, or a combination thereof.

In one embodiment, both the bioreactor vessel 320 and the bioreactor lid 328 are made of resin and formed in a blow-mold, wherein the side wall 336 of the vessel lid is sized such that it can slide into the central opening 326 of the bioreactor vessel, after it has been cut from the top of the bioreactor vessel 320. In one embodiment, the bioreactor vessel is made of steel and a disposable plastic liner is inserted therein.

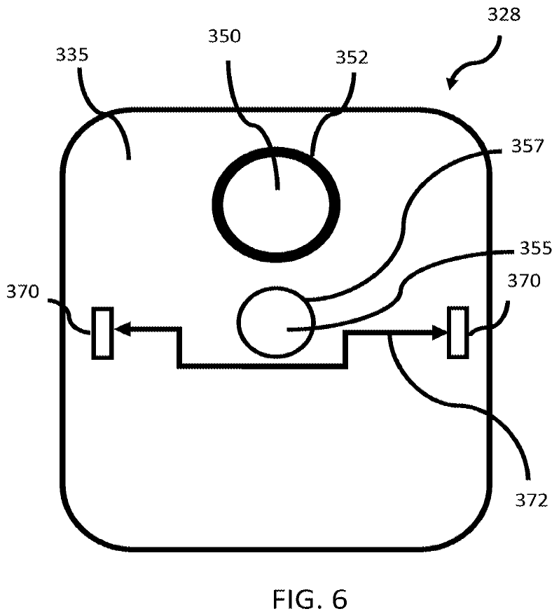

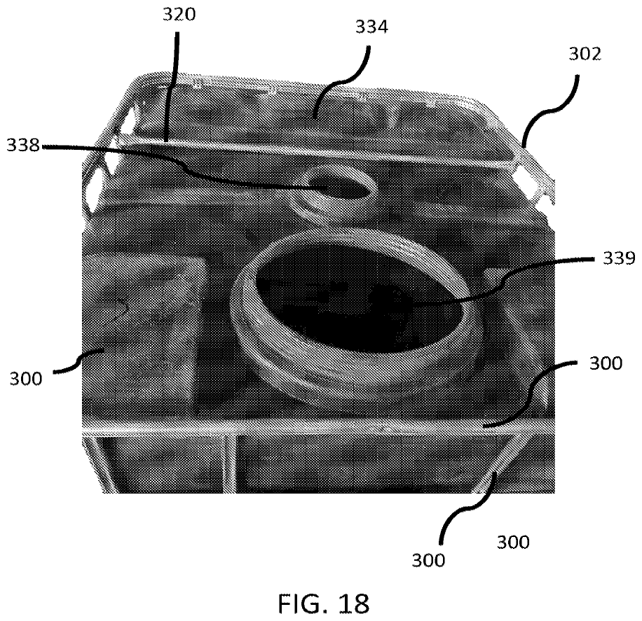

In addition, the top wall 335 of the vessel lid 328 may be provided with one or more ports, such as ports 350, 355 as shown in FIGS. 6 and 18, for accessing an interior of the bioreactor vessel 320 without removing the bioreactor lid 328. According to some embodiments, the ability to remove the vessel lid 328 from the bioreactor vessel 320 allows the vessel 320 to be cleaned and reused for various fermentations, and vessel lids 328 having various port arrangements are selected (or created) based on the needs of the fermentation. The vessel lid 328 optionally comprises a positive displacement seal 338 or other means to ensure a positive air pressure within the bioreactor vessel during fermentation

### Means to Exclude External Contamination

In order to meet sanitation requirements, the bioreactor vessel will be configured to exclude external contamination and the different options will be generally be specified in appropriate regulations. For example, the design and construction of the transportable bioreactor could follow the recommendations of 3-A Sanitary Standards, Inc. In general, food processing operations and retailers must comply with the various HACCP sanitary standards and regulations promulgated by various state and local Health Departments, Food processing operations are certified by a third-party agency who will list the sampling points and provide safety certification. Examples of certifications comprise Good Manufacturing Practices (GMP), HACCP or the International Standards Organization (ISO).

As described herein, with reference to the relevant description of the system, such means can include air locks, positive pressure seals, other means to ensure a positive air pressure within the bioreactor vessel during and after fermentation.

One means to optimize sanitation is to lower the pH within the bioreactor vessel to 4.0 or lower as soon as possible after adding fruit biomass to the vessel. This can be accomplished either through fermentation, adding an organic acid such as acetic acid, and/or citric acid, adding a source of carbon dioxide such as dry ice.

### Scale

In one embodiment, depicted in FIG. 5 the scale of a bioreactor vessel 320 is an 800 Liter container that is, for example, 38 inches wide, 44 inches deep and 33 inches high, with an appropriately sized vessel lid 328. This FIG. and dimensions are not intended to limit the dimensions of the bioreactor vessel 320, but to indicate the general scale of the system. For example, it is not a small-scale bench-top design.

In one embodiment, the transportable bioreactor is configured according to ISO standards, wherein the scale of a cylindrical bioreactor vessel 302 ranges from 17,500 to 26,000 liters (3,800 to 5,700 imp gal; 4,600 to 6,900 U.S. gal) and the outer support structure 302 can be 6.05 meters (19.8556 feet) long, 2.40 meters (7.874 feet) wide and 2.40 meters (7.874 feet) or 2.55 meters (8.374 feet) high.

### Access Ports

In one embodiment, the transportable bioreactor 300 comprises a lower access port 340 as shown in FIGS. 3, 4 & 5. In one embodiment, the transportable bioreactor 300 is designed such that the fruit biomass can be pumped from the bottom of the tank through external tubing into one of the access ports 350, 355, for example, located in the vessel lid 328 of the bioreactor. In one embodiment, a 3-inch ball valve is positioned in a lower access port 340 in the side wall near the bottom of the bioreactor. In one embodiment, a 3-inch butterfly valve is located in a lower access port 340 in the side wall near the bottom of the bioreactor.

In some embodiments, the transportable bioreactor 300, comprises one or more top access ports. In one embodiment, each access port comprises a positive displacement seal or other means for maintaining a positive pressure within the inner container (eg check valve or air lock)

In some embodiments, the vessel lid 328 defines one or more ports through which one or more probes or conduits are insertable into the bioreactor vessel 320. For example a temperature probe and/or a pH-meter probe may be disposed within the bioreactor vessel 320 via port 355 located in the vessel lid 328. In some embodiments, the one or more ports comprises a sample collection port. In one embodiment, the vessel lid 320 has one port in it. In one embodiment, as depicted in FIGS. 6 and 18, the vessel lid 328 of the transportable bioreactor 300 has two access ports in it. In this embodiment, one access port is a central access port 355 with an outer rim 357 and a larger secondary access port 350 with an outer rim 352. In one embodiment, an air-lock means 368 (See FIGS. 4 & 5) is attached to an access port, such as a central access port 355 in order to maintain a positive pressure within the bioreactor vessel 320 to prevent microorganisms and other contaminants to enter into the bioreactor vessel 320.

In one embodiment, an access port, such as a secondary access port 350, for example comprises a screw thread on its outer rim 352, or perimeter to accept a ring or “band”. The band, when screwed down, presses a separate clear disc-shaped lid, such as a clear acrylic disc against the outer rim 352. A ring-shaped seal on the underside of the clear disc-shaped lid creates a hermetic seal around the outer rim 352 of the secondary access port 350 and provides a viewing portal for visually examining the contents of the transportable bioreactor 300.

For example, as shown in FIGS. 3, 4, 5, 6, & 17 the vessel lid 328 comprises a central access port 350 may be included in or attached to the vessel lid 328, providing entry into the bioreactor vessel 320, without having to remove the vessel lid 328. In one embodiment, as depicted in FIGS. 3 & 4, the bioreactor vessel 320 has a lower access port 340 in the wall of the bioreactor vessel 324, providing entry into the lower portion of the bioreactor vessel 320. Other types of sub-systems may be in communication with the interior of the exterior and the interior of the bioreactor vessel 320 via the ports 350 and/or port 355, such as, for example, an exhaust gas collection system. Exhaust gas may be collected for carbon sequestration.

### Aeration Means

The bioreactor vessel is provided with means to aerate the fruit biomass. The objective is to provide sufficient oxygen to force aerobic fermentation and not allow anaerobic fermentation to occur. The rate of fermentation affects flavor of the finished product.

In one embodiment, the bioreactor vessel is also configured to conduct anaerobic fermentation prior to aerating the bioreactor vessel and conducting aerobic fermentation. Typically, tanks are vented, so a typical vent is spring loaded so that there is a minimal amount of pressure on the spring. If the internal tank pressure is too low or too high, the air lock will vent.

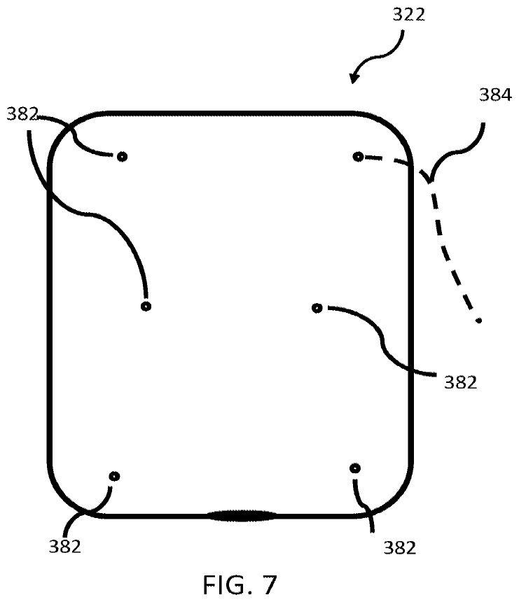

In one embodiment, the transportable bioreactor 300 has aeration means 380, which may or may not be attached to the transportable bioreactor 300. For example, one embodiment illustrated in FIG. 7, comprises a number of aeration ports 382 located in the bottom surface 322 of the bioreactor vessel 320. Aeration lines 384 may be attached to the aeration ports 382 on the bottom surface 322 a transportable bioreactor 300 and an aeration means 380 attached to the gas lines to provide air to the one or more transportable bioreactors 300. In one embodiment depicted in FIG. 3, the transportable bioreactor 300 has an aerator 360, attached to support means 362, wherein the aeration means gains access to the biomass through a lower access port 340, and forces air into the lower area of the bioreactor vessel 320 allowing air to percolate up through the mixture, encouraging aerobic fermentation. The aerator 360 could either be powered via external electric power means 364 or solar/battery power (not shown).

In one embodiment, a metered amount is provided daily, for example, 25 Liters of air/day to aerate 800 liters of material. In one embodiment, an optimal oxygen diffusion amount is achieved and maintained throughout the fermentation process.

### Appropriate Pressure Within the Bioreactor Vessel

In one embodiment, the bioreactor vessel 324 is designed to maintain a positive air pressure within the vessel and thereby comprises positive pressure maintenance means. For example, transporting a bioreactor vessel 324 to a fruit processing facility, which is a non-sterile environment, particular care might be taken to ensure that ingress and/or contamination of microbes, dust, pollen, insects, spores, toxic substances, etc. is minimized so as to not contaminate the fruit biomass. If the bioreactor vessel 324 is scaled, and a gas such as air is being delivered into the bioreactor vessel, one or more exhaust ports on the vessel can be maintained at any pressure desirable for the specifications of the particular application of the system by including a pressurized valve on the exhaust port(s). Another example of positive pressure maintenance means comprises, a valve, comprising a ball & spring valve.

A check valve is a valve is a valve that normally allows fluid (liquid or gas) to flow through it in only one direction. Check valves are two-port valves comprising two openings in the body, one for fluid to enter and the other for fluid to leave. There are various types of check valves used in a wide variety of applications, also known as a non-return valve, reflux valve, retention valve, foot valve, or one-way valve and are often part of common household items.

For example a simple air pump, such as commonly used with fish tanks, is always protected by a check-valve, in which the air pushes the valve open, while the pump is operating, but the size of the piston inside the check-valve is such that if the air pressure stops, the piston returns to close the opening and prevents water from flowing into the pump.

In one embodiment, an airlock 368, such as illustrated in FIG. 4 can be inserted into a port, for example, a central port 355, as illustrated in FIG. 6. There are a wide variety of fermentation locks or air locks, one of which can be selected and inserted into a port to manage an appropriate positive pressure within the bioreactor, balanced against not allowing too much pressure to build up within the vessel. A fermentation lock allows carbon dioxide released during fermentation to escape the vessel, while not allowing air to enter, thus avoiding oxidation. This lock is generally in the form of a tube or tube-like structure connected to the headspace of a fermenting vessel in fluid communication with a container of sanitized liquid, eg., water which may comprise Sulphur dioxide or alcohol to prevent contamination of the vessel contents if the liquid is inadvertently drawn into the vessel. When the pressure of the gas inside the vessel exceeds the prevailing atmospheric pressure the gas will bubble through the liquid towards the outside air.

### Optional Mixing Means

In certain embodiments, each container can contain, either partially or completely within its interior, an impeller assembly for mixing, dispersing, homogenizing, and/or circulating one or more liquids, gases and/or solids contained in the container. In accordance with certain embodiments, the impeller assembly may include one or more blades or vanes, which are movable, such as by rotation or oscillation about an axis. In certain embodiments, the impeller assembly converts rotational motion into a force that mixes the fluids it is in contact with. In certain embodiments, the blades are made of plastic.

### Optional Pump-Grinder Means

In one embodiment, a pump-grinder is connected to each transportable bioreactor 300 via external tubing or hose such that fruit biomass can be drawn out of the bottom of the bioreactor vessel 320 and ground as it passes through the tubing/hose and fed into into the top of the bioreactor vessel 320. In one embodiment, a pump-grinder can be removably attached to the external tubing of each bioreactor. In one embodiment, there is a common pump-grinder that is used for each of the transportable bioreactors 300 in the fruit processing facility. In one embodiment, a pump-grinder could be shared between a number of fruit processing facilities.

The timing and sequence of events following transfer of fruit biomass into one or more transportable bioreactor(s) 300 can vary, depending on the type of fruit biomass, the location of the fruit processing facility and the location determined to initiate and at least partly allow for the fermentation of the fruit biomass.

For example, in one embodiment, the fruit biomass is passed through a mill such as a hammer mill or a brush mill and separating out seeds, pits, skins, etc., prior to transferring the remaining fruit biomass to the bioreactor vessel 320. In one embodiment, the fruit biomass is ground into a course paste to expose as much surface to microbes as possible. In one embodiment grinding the fruit biomass should be performed as early in the process as possible, but might not be done prior to initiating the fermentation.

In one embodiment, the fruit biomass is fully re-hydrated prior to grinding. In one embodiment, the fruit biomass is ground prior to re-hydrating it. In one embodiment, the fruit biomass is ground and the fermentation initiated without fully re-hydrating the fruit biomass. In one embodiment, the fruit biomass is ground and fermented without adding any water.

In one embodiment, the system comprises one or more permanent or transferable in-container mixer/homogenizer. In one embodiment, the system comprises one or more external pump-over homogenizer(s). In one embodiment, the system comprises one or more in-container pasteurization means (similar in design to a homogenizer but has a heat-exchanger built into the rods). In one embodiment, the system comprises one or more external pump-over pasteurization means. In one embodiment, the system comprises a mixing disperser emulsify homogenizer high shear mixer. In one embodiment, the system comprises the incorporation of ultrasonic food processing at the beginning of the process to grind the berries, and at the end of the fermentation process to inactivate the microbes.

### Optional Carbon Dioxide Sequestration Means

As a result of growing concern over the potentially devastating effects of global warming, the United Nations Framework Convention on Climate Change (UNFCCC) was adopted in 1992. The Kyoto protocol, which is a protocol to the UNFCCC, was in Kyoto, Japan, on 11 Dec. 1997. The provisions of the Kyoto protocol attempt to regulate the output of carbon dioxide by member states that have signed and ratified the protocol. The UNFCCC allows for a system of carbon trading. Under this system, parties who establish carbon sinks obtain a “carbon credit” in respect of the amount of carbon dioxide taken up into the carbon sinks. This carbon credit can be traded to greenhouse gas emitters in order to enable the emitters to meet their targets under the Kyoto protocol.

Carbon is sequestered in soil by plants through photosynthesis and can be stored as soil organic carbon (SOC). Agroecosystems can degrade and deplete the SOC levels but this carbon deficit opens up the opportunity to store carbon through new land management practices. Soil can also store carbon as carbonates. Carbonates are inorganic and have the ability to store carbon for more than 70,000 years, while soil organic matter typically stores carbon for several decades. Scientists are working on ways to accelerate the carbonate forming process by adding finely crushed silicates to the soil in order to store carbon for longer periods of time.

In one embodiment, this system comprises one or more container airlock with CO2 exhaust hose that can be buried in a fruit processing facility for CO2 sequestering. Incorporating this option into the system, add another potential “revenue stream” for fruit processing facilities and other businesses participating in the business model.

### Optional Support Module

One embodiment comprises a support module as illustrated in FIG. 17, where various instruments and machines such as an aerator 360 air pump, instrument interface, data logger 525, power source 364 (battery pack, generator, solar power means), security means (e.g. camera), weather station, RFID, and other such supporting devices can be located and used to support the use of one or more transportable bioreactor.

### The Monitoring Subsystem

One embodiment of the system does not comprise a monitoring system. In this embodiment, the progress of the fermentation reaction may or may not be monitored. For example, in environments where the temperature is cold or very cold, the reaction would proceed so slowly that it may not be necessary to monitor the bioreactor vessel 320. In another example, the transportable bioreactor 300 may be transferred to a fruit processing facility for collection of fruit biomass, without initiating fermentation at the fruit processing facility. These transportable bioreactors 300 may be transported to another location, wherein one or more individuals could initiate fermentation therein. Again, if the temperature is cold, it may not be necessary to monitor the progress for an extended period of time.

In the most basic manner, monitoring could entail an individual walking around with a hand-held thermometer and/or a pH meter and manually measuring the relevant qualities of the fruit biomass within the bioreactor vessel 320. Thus, one embodiment of this system comprises monitoring by an individual using one or more hand-held devices, such as a pH monitor and/or a thermometer. Depending on the design of the system, it may be preferable to place the sensors into the headspace only of the bioreactor vessel. In one embodiment, it may be preferable to place the sensors directly into the fermenting fruit biomass to take a direct reading of the biomass, with appropriate sanitation methods.

One embodiment of the system optionally comprises one or more sensor devices to monitor one or more qualities pertaining to the transportable bioreactor 300 such as temperature (both within the bioreactor and external to the bioreactor), and pH level of the fruit biomass within the bioreactor vessel 320.

In general, it is important to bring the pH of the fruit biomass down to a level at or below pH 4.0 as soon as possible after adding fruit biomass to the bioreactor vessel. In one embodiment, the system includes means to measure volatile organic compounds (VOC's) in the headspace of the bioreactor vessel and to interpret the data to track fermentation. VOC's comprise any volatile compound present in the bioreactor and the headspace composition will be in equilibrium with the bioreactor contents. The partial pressure for each compound will indicate the progress of the fermentation. In one embodiment, the system comprises means to detect and quantify these compounds in order to control the process.

Without being limited or bound to a specific embodiment, there are a number of different sensors that could be included in the optional monitoring subsystem to monitor the progression of fermentation of the fruit biomass within the bioreactor vessel 320, including Brix (sugar levels), ion specific (lactate, malate, acetate, potassium, sulfite, etc.), refractometric, optical density, colourimetric, temperature, density, pH, moisture, etc. As technologies evolve, there will be additional sensory methods that can be deployed to monitor the fermentation process. There are immersed sensors, including are pH, dissolved oxygen, temperature, refractive index, conductivity, and ion selective electrodes such as sulfite or lactate. Other types of sensors detect optical density or colour (hue and intensity), or NIR, sometimes called FTIR (Fourier Transform Infra-Red). Gas sensors, used for headspace analysis would be for particulates, including aerosols and volatile organic compounds (VOCs). A method such as Near Infrared (NIR) should be able to individual compounds within VOCs.

For example, a temperature probe and/or a pH-meter probe may be disposed within the bioreactor vessel 320 via port 355 located in the vessel lid 328 and optionally is in communication with a data logger 525, which is disposed outside of the bioreactor vessel 320. The system is designed to prevent clogging, flowing and coating of a sensor immersed in the fruit biomass.

In one embodiment, for example, a small well can be immersed, one that contains a membrane to exclude solids from filling the well. The liquid in the well should contain the same composition of solutes and can be monitored without damaging the electrodes with the solid material.

In one embodiment, one or more sensors could be attached to the bioreactor 300, and could optionally be monitored by using Wi-Fi. In one embodiment, one or more sensors are not attached to the bioreactor, but are used by an individual tasked with monitoring the progress of fermentation within the one or more bioreactors. The optional monitors could either be powered via external electric power or solar/battery power.

In one embodiment, the bioreactor 300 comprises a monitoring system comprising one or more sensing means that can be located in the headspace of each bioreactor. In one embodiment, one or more sensors may be able to be inserted directly into the fruit biomass, such as a temperature monitor.

It is important to stop the fermentation reaction at the point where the levels of acetic acid and lactic acid are optimal for the eventual product and not to let the fermentation to go to completion, wherein the acetic acid and lactic acid are converted to CO₂ and H₂O. In one embodiment, the use of appropriate sensors located in the headspace of the bioreactor vessel, would assist in the determination of optimal timing to cease the fermentation process.

In one embodiment, the fermentation process is regulated by the temperature in the bioreactor.

In one embodiment, the fermentation process is regulated by using the amount and rate of aeration (oxygen diffusion).

In one embodiment, the fermentation process is simply allowed to proceed given the natural conditions in the environment and the process is monitored to determine when the fermenting biomass is getting near completion.

One embodiment of the monitoring sub-system comprises an RFID tag to identify each bioreactor and to be able to track its location, for example using GPS. This information might also assist with product traceability (keeping detailed records of where all raw material comes from and who products are sold to), which is mandatory in most jurisdictions. The information provided by an RFID tag on a bioreactor could be particularly important in larger processing facilities where bioreactors could come in from a number of fruit processing facilities.

Generally stated, radio-frequency identification is the use of electromagnetic energy to stimulate a responsive device (known as an RFID) “tag” or transponder) to identify itself and, in some cases, provide additional information and/or data stored in the tag. RFID tags typically comprise a semiconductor device commonly referred to as the “chip”, upon which are formed a memory and an operating circuitry, which is connected to an antenna. Typically, RFID tags act as transponders, providing information stored in the chip memory in response to a radio frequency interrogation signal received from a reader, also referred to as an interrogator. In the case of passive RFID devices, the energy of the interrogation signal also provides the necessary energy to operate the RFID tag device. RFID tags are manufactured with a unique identification number which is typically a simple serial number of a few bytes with a check digit attached. This identification number is typically incorporated into the RFID tag during its manufacture. The user cannot alter this serial/identification number, and manufacturers guarantee that each RFID tag serial number is used only once and is, therefore, unique. Such read-only RFID tags typically are permanently attached to an article to be identified and/or tracked and, once attached, the serial number of the tag is associated with its host article in a computer database.

In some cases, the RFID tag may be attached to the outside of the transportable bioreactor with a clip, adhesive, tape, or other means and, in other cases, the RFID tag may be inserted within the structure of the transportable bioreactor in, a manner that it can not be removed or tampered with. Further,

In one embodiment, a security means is attached to the top lid(s) to prevent unauthorized entry into a bioreactor.

### Optional Temperature Moderating Means

In one embodiment temperature moderating means may be included in the system. For example, heat exchangers may be incorporated into the system. In one embodiment, the air used to aerobically ferment the fruit biomass may be heated or cooled. The specifications required for fermentating in the local environment will determine whether heating or cooling the bioreactor vessel will be beneficial.

### Optional Insulating Means

In one embodiment, the system comprises a vessel insulated wrapper, such as a silver wrapper, that can be wrapped around one or more bioreactors to assist in maintain the temperature within the bioreactor and its contents above freezing conditions. In one embodiment, an insulated wrapper can be sized to cover five containers at the same time.

Some of the advantages of incorporating one or more insulating wrapping means within the system is to: protect the containers from undetected tampering (resistant to penetration); hold internal heat and absorb solar heat (insulation/dark matte finish); and minimize visibility (esthetics, avoiding vandalism).

### The Optional Network

With reference to FIG. 7, the network would provide periodic information from each bioreactor to the producer, the local business and to the international business. This information may be used to make realtime decisions on when to transport biomass to production facility; to discover corelations between temperature, terroir, varital, and characteristics of specific biomass; and to project potential supply-line of specific products.

### The Design of the Networked System

In one embodiment, the bioreactors are monitored remotely through a sensor subsystem in addition to be monitored on site at the fruit processing location. In one embodiment, the system is a networked system 500.

Referring to FIG. 7, a simplified block diagram of an exemplary embodiment of a networked system 500. The networked system 500 and method includes one or more bioreactors 300 with one or more sensors positioned in the headspace of the bioreactor 300 and optionally in the environment of the fruit processing facility, a company computer system 540, a computer system with database 570, and a plurality of wireless monitoring devices 544, which may be communicatively connected through a network 510, as described below. In one embodiment, the networked system 500 and the monitoring devices 544 may communicate via wired and/or wireless signals over a communication network 510, which can be any suitable local or wide area network(s) including a WiFi network, a Bluetooth network, a cellular network such as 3G, 4G, Long-Term Evolution (LTE), 5G, the Internet, etc. In some instances, the monitoring devices 544, may communicate with the communication network 510 via an intervening wireless or wired device, which may be a wireless router, a wireless repeater, a base transceiver station of a mobile telephony provider, etc.

The monitoring devices 544 may include, by way of example, a hand-held device, a tablet computer, a smart watch, a network-enabled cell phone, a wearable computing device, a personal digital assistant (PDA), a mobile device smart-phone also referred to herein as a “mobile device.” a laptop computer, a desktop computer, a phablet, any device configured for wired or wireless RF (Radio Frequency) communication, etc.

In some embodiments, personnel at the fruit processing facility may enter data into the desktop computer 546 for example, such as information relevant to traceability requirements.

Each of the sensors located in a bioreactor 300 may interact with the networked system 500 to transmit data pertaining to the environment in addition to the fermentation conditions as measured in the headspace of a bioreactor. In some embodiments, environmental and fermentation data may be collected periodically (e.g., every hour, every day, every week, etc.) to identify changes to the environment and fermentation conditions over time. In some embodiments, environmental and fermentation data may be collected continuously or nearly continuously to identify changes to the environment and fermentation conditions over time

In one embodiment, a data logger 525 located within a data store of the network, on a local workstation 540 and/or on the network computer 520 stores data collected from the bioreactor vessel 320. For example, data logger 525 may store data such as ph levels, oxidation-reduction potential, and/or temperature of the liquid medium in the bioreactor vessel 320. In some embodiments, an appropriate data measurement system is in electrical communication with the data logger 525 and provides the data to the data logger 525. The data logger 525 is also configured for communicating at least a portion of the stored data to a computing device, according to some implementations. The data logger 525 provides more flexibility in the type of data collected from the contents of the bioreactor vessel 320 and how the collected data is stored and used. In some configurations, the data logger 525 could also be a computer.

A “computer” or “computing device” may refer to one or more apparatus and/or one or more systems that are capable of accepting a structured input, processing the structured input according to prescribed rules, and producing results of the processing as output. Examples of a computer or computing device may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

“Software” or “application” may refer to prescribed rules to operate a computer. Examples of software or applications may include: code segments in one or more computer-readable languages; graphical and or/textual instructions; applets; pre-compiled code; interpreted code; compiled code; and computer programs.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hypertext Markup Language (HTML.), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Go, Java™, C, C++, Smalltalk, Python, Perl, UNIX Shell. Visual Basic or Visual Basic Script, Virtual Reality Markup Language (VRML), ColdFusion™ or other compilers, assemblers, interpreters or other computer languages or platforms.

Computer program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). The program code may also be distributed among a plurality of computational units wherein each unit processes a portion of the total computation.

The Internet is a worldwide network of computers and computer networks arranged to allow the easy and robust exchange of information between computer users. Hundreds of millions of people around the world have access to computers connected to the Internet via Internet Service Providers (ISPs). Content providers (e.g., website owners or operators) place multimedia information (e.g., text, graphics, audio, video, animation, and other forms of data) at specific locations on the Internet referred to as webpages. Web sites comprise a collection of connected, or otherwise related, webpages. The combination of all the web sites and their corresponding webpages on the Internet is generally known as the World Wide Web (WWW) or simply the Web.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically, a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random-access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASHEEPROM, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G, 4G and the like.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory or may be communicated to an external device so as to cause physical changes or actuation of the external device.

### Blockchain for Security

In one embodiment, the privacy issues pertaining to the data and information could be protected by using blockchain technology. A blockchain is also referred to as a distributed ledger, that is, a transaction is jointly accounted by a plurality of nodes distributed at different places, and each node records a complete account. In a narrow sense, the blockchain is a chained data structure obtained by combining data blocks in a chronological order, and a distributed ledger that is protected against tampering or forging by using cryptology.

For a blockchain technology, the blockchain has features of being decentralized and trustless. Decentralized means that there is no centralized hardware or management mechanism in an entire blockchain network, rights and obligations of nodes are equal, and damage or loss of any node does not affect operation of an entire network system. Therefore, desirable robustness of the blockchain network is ensured. Trustless means that nodes do not require mutual trust to participate in data exchange in the entire network. An operation rule of the entire blockchain network system is open and transparent, and data content on all blockchain nodes is also open. Therefore, within a rule or a time range specified by the system, the nodes cannot cheat each other.

To protect important information, utilizing storage on cloud networks is one approach to provide data redundancy. For sensitive information, the information may be stored in an encrypted form. Blockchain leverages both cloud networks and encryption to define storage of all information in a block wise manner. The blocks are added to the blockchain in a linear and chronological order. The blockchain helps to store and track data in a secured manner.

A client requests access data in a node on a blockchain network, and the node on the blockchain network provides encrypted data and a decryption key for the client. Then, the client decrypts the encrypted data by using the decryption key to obtain the access data, and records the access data on the blockchain.

Examples described herein extend to validation of any common data access transactions, where the proof of verification protocol may be used to validate an owner of data before a transaction is granted. Common data access transactions may comprise but are not limited to: transactions that request access to user data, transactions that request sharing of user data, transactions that request transfer of ownership of user data, transactions that request redemption of currency or rewards associated with a user account, transactions that modify user data and transactions that monitor user activity/application data, among other examples.

In some examples, exemplary proof of verification processing is used to validate what may be considered new common data access transaction requests, which do not attempt to alter a record of a distributed ledger of previously validated transactions. If a subsequent request is received to retroactively alter a block of the distributed ledger, an exemplary blockchain layer implements a blockchain protocol to prevent retroactive alterations to data of validated transactions. For instance, upon generation of a block, cryptographic hashing is used to lock blocks of the distributed ledger. Attempts to alter a created block (in distributed ledger) would result in a new hash being generated that would not match the hashing in that block or subsequently generated hashes from that block. Proof of verification processing may further be applicable as an alternative or supplement to other proof protocols such as proof-of-work and proof-of-stake, among other examples.

The present disclosure is applicable to any distributed networking (e.g., cloud-computing network) instances where data is managed in a decentralized manner. Examples described herein apply to IoT examples such as instances where a number of smart devices are connected to manage data and operations in a close geographic proximity (e.g., home). Exemplary proof of verification processing may be utilized to validate data access transactions including control of a specific smart device in an IoT network. In another example, an entity may establish a distributed storage that stores data, which can be accessed via a network connection. The distributed storage is managed across a plurality of nodes, which may be virtualized computing nodes. The plurality of nodes, or a separate set of (virtualized) computing nodes, may manage validation of common data access transactions with respect to data that is stored across the distributed storage. A proof of verification protocol is applied to validate propriety of access to data for any of the described common data access transactions. Stored data may be encrypted to add an additional layer of security however access application of the proof of verification focuses on access request to the data. A distributed ledger is utilized to manage recordation of all validated common data access transactions. In some examples, an exemplary distributed ledger may be managed internally by a specific entity, company, business, etc., for example, for records management and auditing purposes. Additionally, the distributed ledger may be accessible to manager of protected data, for example, a subscriber (e.g., user) of an application/service who creates or own user data. In such instances, subscribers may access a record of common access data transaction relating to their user data.

### Making a Bioreactor

There are a number of possible containers known in the industry that would be appropriate candidates for conversion into a bioreactor, depending on the volume demands of a fruit processor. These include but are not limited to plastic cylinder tanks and bulk wine shipping bladders.

Making a Bioreactor from Intermediate Bulk Container

In one embodiment, the bioreactor can be a rigid intermediate bulk container (IBC), which are also known as an IBC tote, IBC tank, IBC, or pallet tank, which has been adapted to conduct anaerobic fermentation.

Rigid IBCs are stackable, reusable, versatile containers with an integrated pallet base mount that provides forklift and/or pallet jack maneuverability. Most IBC's are cube-shaped and this cube-shaped engineering contributes to the packaging, stacking, storing, shipping, and overall space efficiency of intermediate bulk containers. Almost all rigid IBCs are designed so they can be stacked vertically one atop the other using a forklift.

The support structure/containers can be made from metal (stainless steel), plastic (high-density polyethylene), or a composite construction (galvanized steel and plastic) of the two materials. The IBC tank can be made of plastic, stainless steel, and carbon steel tanks The IBC tank capacities generally used are often 1,040 and 1,250 litres (275 and 330 US gal).

The most widely utilized and known IBC is the limited re-use, caged IBC tote container. Caged IBC totes are composite intermediate bulk containers—a white/translucent plastic container (typically high-density polyethylene) contained and protected by a tubular galvanized steel grid, common. Most have a built-in tap (valve, spigot, or faucet) at the base of the container to which hoses can be attached, or through which the contents can be poured into smaller containers.

The advantage of modified IBC totes is that most wineries already use unmodified totes and have equipment that allows them to move and stack them on their property. The modified totes can be sealed to minimize food contamination and placed in an external part of the property, either in or adjacent to the vineyard. One of the advantages of in-vineyard placement is CO² sequestering by the vines and undergrowth.

In one embodiment, the bioreactor 300 is a version of a food-grade intermediate bulk container (IBC), commonly referred to as an IBC tote, with a holding capacity of about 1,040 or 1,250 litres (275 and 330 US gal), capable of being stacked, which has been appropriately modified for use with the methods and processes within this system 200.

There are a number of processes for generating the bioreactor vessel 320 of a transportable bioreactor 300, such as rotational casting, injection molding and blow molding among others.

One method that is very well known in the art is rotational casting or molding processes, which are also called rotomolding processes, for the production of hollow plastic articles of different kinds. They are applicable in an economic manner for the production of hollow plastic articles for divers applications, e.g. as tanks, for transportation, as containers, as toys and leisure articles, for materials handling, in the marine industry, for medical or industrial products, etc. In particular the production of large and very large vessels and the production of hollow articles with complex three-dimensional shapes is possible under high quality standards.

In a typical rotomolding process as known in the art, a known amount of a polymeric compound which may be in powder, granular or liquid form is charged into a hollow, shell-like mold (rotomold). The mold is then heated and simultaneously rotated about two principal axes so that the polymeric compound enclosed in the mold adheres to the inner mold surface and forms a plastic layer thereon. The mold rotation continues during the cooling phase which follows the heating phase so that the plastic layer achieved on the inner surface of the mold retains the desired shape as it solidifies. When the plastic is sufficiently rigid, the mold rotation is stopped and the plastic product may be removed from the mold. The process is executed under a relatively low rotational speed of about 4 to 20 revs/min. Several types of machines bearing the molds may be used, which allow the production steps of mold charching, mold heating, mold cooling and part ejection to be executed simultaneously with different molds in different zones or one by one with more than one mold at a time. In addition, the motion in two principal axes may be completed by a “Rock and Roll” motion as well.

Usually a rotomold is composed of two parts which are clamped together to form the hollow rotomold when the rotomolding process is executed, but rotomolds which are composed of three or more parts may also be used. Rotomolds with relatively thin wall thickness are possible, since the rotational molding process is, in most cases, executed under atmospheric pressure or, in exceptional cases, under only small pressure or vacuum. The rotomolding process allows the production of hollow plastic articles with uniform wall thickness, having neither pinch-off seams nor welding lines.

Injection molding is a manufacturing process for producing one or more parts by injecting molten material into a mold. Injection molding can be performed with a host of materials mainly including metals (for which the process is called die-casting), glasses, elastomers, confections, and most commonly thermoplastic and thermosetting polymers. Injection molding is well known in the art.

Material for a part is fed into a heated barrel, mixed, and injected into a mold cavity, where it cools and hardens to the configuration of the cavity. After a product is designed, usually by an industrial designer or an engineer, molds are made by a mold-maker from metal, usually either steel or aluminum, and precision-machined to form the features of the desired part.

Parts to be injection molded must be very carefully designed to facilitate the molding process; the material used for the part, the desired shape and features of the part, the material of the mold, and the properties of the molding machine must all be considered. The versatility of injection molding is facilitated by this breadth of design considerations and possibilities.

When plastic totes are manufactured via conventional injection molding, the mold includes injection gates on the bottom portion of the tote in the mold. These bottom gates supply plastic melt to the entire mold. These systems require relatively large amounts of clamp tonnage and injection pressure to ensure the mold is completely filled. The resulting plastic tote has relatively high stress points on the bottom walls, which leads to a less durable final product.

Blow molding is a manufacturing process for forming and joining together hollow plastic parts such as bottles or other hollow shapes. There are three main types of blow molding: extrusion blow molding, injection blow molding, and injection stretch blow molding.

Blow-molded containers are usually produced by expanding a preform made of a thermoplastic material such as PET into a blow mold by applying blow pressure and then filling it with a product (especially a fluid) in a subsequent filling station. It is also known that a preform is expanded into a blow mold by the pressure of a fluid to be filled, so that forming and filling represent a common method (also called “formfill”).

### The Blow Mold

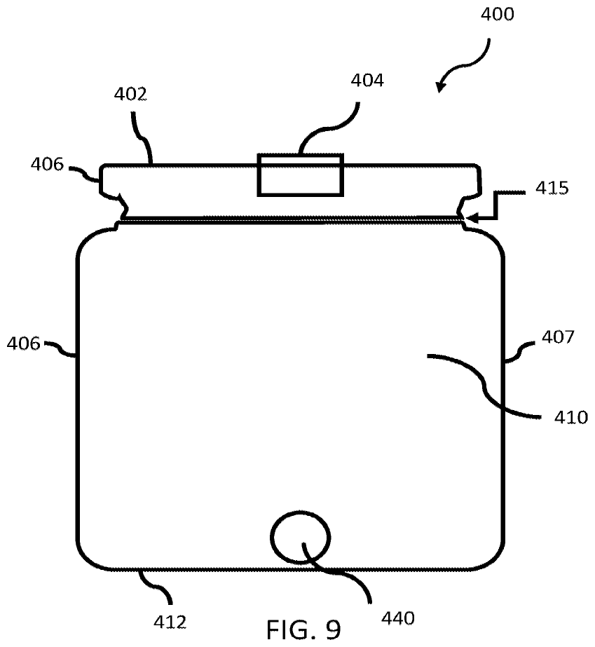

One non-limiting example for how to make a bioreactor vessel is described using blow mold technology. One embodiment of a blow mold design, the front face of which is shown in FIG. 9.

### The Microbial Formulation

### Acetic Acid Bacteria

The steps of the derivative-conversion require inoculation of microbial formulation, comprising acetic acid bacteria. In accordance with the present invention, elements of the microbial formulation are selected from the family of family Acetobacteraceae.

Acetic acid bacteria (AAB) are a group of rod-shaped, Gram-negative bacteria which aerobically oxidize sugars, sugar alcohols, or ethanol with the production of acetic acid as the major end nutrient-rich product. This special type of metabolism differentiates them from all other bacteria. The acetic acid bacteria consist of 10 genera in the family Acetobacteraceae, including Acetobacter. Species of Acetobacter include: A. aceti; A. cerevisiae; A. cibinongensis; A. estunensis; A. fabarum; A. farinalis; A. indonesiensis; A. lambici; A. liquefaciens; A. lovaniensis; A. malorum; A. musti; A. nitrogenifigens; A. oeni; A. okinawensis; A. orientalis; A. orleanensis; A. papaya; A. pasteurianus; A. peroxydans; A. persici; A. pomorum; A. senegalensis; A. sicerae; A. suratthaniensis; A. syzygii; A. thailandicus; A. tropicalis; and A. xylinus. Several species of acetic acid bacteria are used in industry for production of certain foods and chemicals.

The strains, which have been identified include: Acidibrevibacterium Acidicaldus Acidiphilium Acidisoma Acidisphaera Acidocella Acidomonas Ameyamaea Asaia Belnapia Bombella Caldovatus Commensalibacter Craurococcus Crenalkalicoccus; Dankookia Elioraca Endobacter Gluconacetobacter; Gluconobacter Granulibacter Humitalea Komagatabacter Komagataeibacter Kozakia Muricoccus Neoasaia Neokomagataed Nguyenibacter Paracraurococcus; Parasaccharibacter. Although a variety of bacteria can produce acetic acid, mostly members of Acetobacter, Gluconacetobacter, and Gluconobacter are used commercially.

### Lactic Acid Bacteria

Lactic acid bacteria (LAB) are an order of gram-positive, acid-tolerant, generally nonsporulating, non=respiring, either rod-shaped (bacilli) or spherical (cocci) bacteria that belong to the order Lactobacillus and share common metabolic and physiological characteristics. Lactic acid bacteria are used in the food industry for a variety of reasons such as the production of cheese and yogurt nutrient-rich products. The genera that comprise the LAB are at its core Lactobacillus, Leuconostoc, Pediococcus, Lactococcus, and Streptococcus, as well as the more peripheral Aerococcus, Carnobacterium, Enterococcus, Oenococcus, Sporolactobacillus, Tetragenococcus, Vagococcus, and Weissella.

### Yeasts

When the fruit biomass comprises a relatively high sugar content, then an oxygen-tolerating yeast may be included in the microbial formulation. Fermentation normally occurs in an anaerobic environment, however, some strains of yeast such as Saccharomyces cerevisiae can be used to ferment the sugar.

Some species of yeast (e.g., Kluyveromyces lactis or Kluyveromyces lipolytica) in the presence of oxygen will oxidize pyruvate completely to carbon dioxide and water in a process called cellular respiration and will only produce ethanol in an anaerobic environment. This phenomenon is known as the Pasteur effect. The Pasteur effect occurs in the presence of moderately high concentrations of sugar. At low levels (e.g., 20 g/L.), yeast can completely metabolize sugar or alcohol to water and carbon dioxide. The Pasteur effect is an essential part of winemaking but will not inhibit wine spoilage in the presence of small amounts of residual sugar. Acetic acid inhibits yeast.

The counter-Pasteur effect is observed with many yeasts such as Saccharomyces cerevisiae or fission yeast Schizosaccharomyces pombe, which under certain conditions, ferment rather than respire even in the presence of oxygen. These yeasts will produce ethanol even under aerobic conditions, if they are provided with the right kind of nutrition.

### Terminating the Fermentation Process

The fermentation process is terminated when the levels of acetic acid and/lactic acid reach a pre-determined composition within the bioreactor vessel. This can be accomplished by sealing the vessel in a manner such that no oxygen can enter the bioreactor vessel. In one embodiment, the process is terminated by additionally sparging with nitrogen, argon, carbon dioxide or a combination thereof.

### System Installation and Use in a Fruit Processing Facility

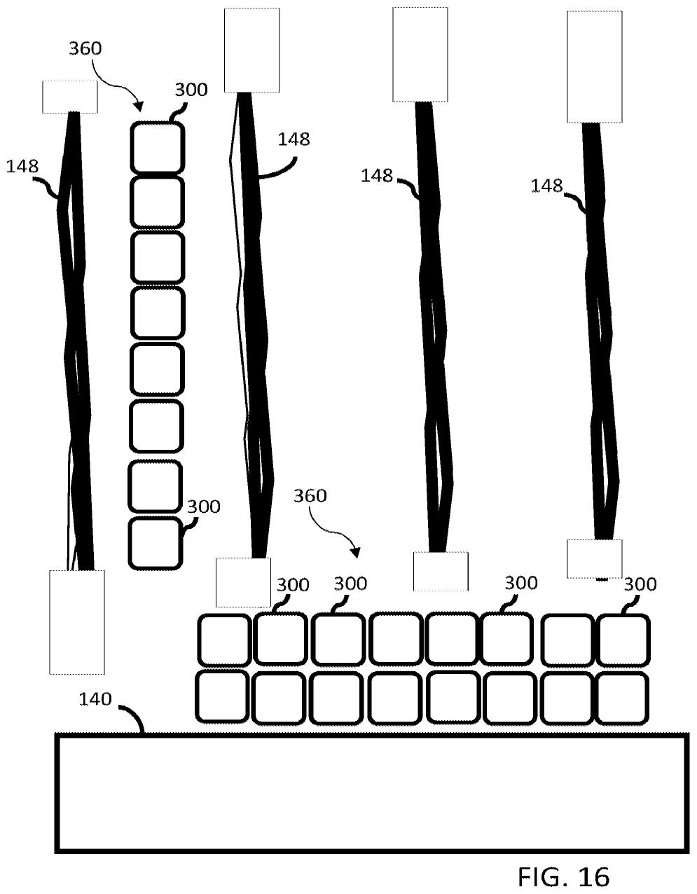

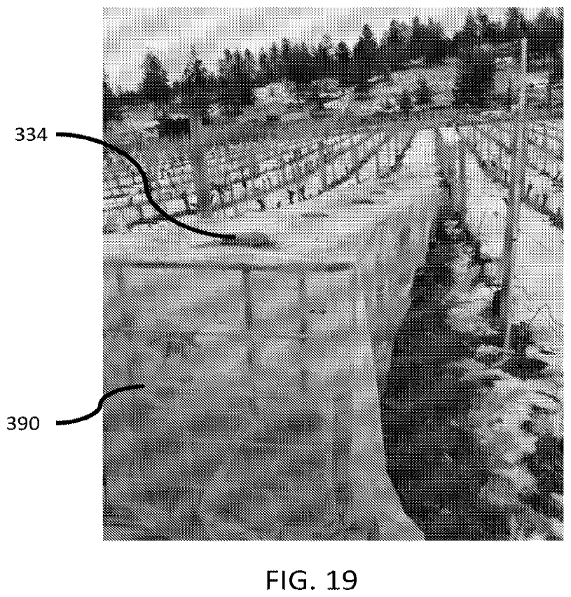

In one embodiment, the system can be transported to a fruit processing facility such as a winery or an orchard, such that the fruit, pomace and/or biomass that would normally be discarded or constitute a lower value stream such as compost or livestock feed, can be placed into the bioreactor on site, and fermentation can be initiated and conducted on location. For example, FIGS. 16 and 19 illustrate the placement of transportable bioreactors at a fruit processing facility, such as a winery, for the initiation and some term of the fermentation process.

Alternatively, once filled, the bioreactors can be transported to another location, such as a field, parking lot, warehouse, etc. where fermentation can be initiated and conducted with minimal supervision. When it is determined appropriate, the transportable bioreactors can be transported to a processing facility, wherein the fermented can be further processed, refined and/or stabilized for use as a food additive, health supplement, or other such product.

### Orchard Deployment

One embodiment of the system is deployed in a fruit orchard, for example an apple or a peach orchard. If the orchard is an old orchard, it likely has large, mature trees which were planted and spaced to accommodate heavy equipment. If the orchard is a modern industrial orchard, it may have been planted in a higher density fashion and may include sensing equipment (“smart orchards”) to monitor the growth of the fruit trees.

One embodiment of using the system comprises the steps of:
a) delivering one or more bioreactors to an orchard;
b) transferring fruit biomass to one or more bioreactors;
c) inoculating the fruit biomass with a microbial formulation;
d) fermenting inoculated fruit biomass to generate fermenting biomass;
e) monitoring the fermentation until it reaches a target stage;
f) optionally performing one or more pre-processing steps and
f) transferring the bioreactor to a processing facility for further processing.
   Possible Variations on this General Process Include:

The pre-processing steps that could be performed at the orchard include:
a) separating the fermented biomass from solid fruit material, such as seeds, stems, skin, etc;
b) adding water

The steps that could be performed at the processing facility include:
a) separating the fermented biomass from solid fruit material, such as seeds, stems, skin, etc to generate an isolate;
b) emulsifying the isolate to generate a puree;
h) optionally inoculating the puree;
i) optionally fermenting said puree to generate a fermented puree;
j) refining fermented puree to generate a refined nutrient-rich product;
k) optionally stabilizing refined product to generate stabilized nutrient-rich product; and
l) optionally packaging the stabilized nutrient-rich product.

### Winery Producing Red Wine

One embodiment of the system is deployed in a winery that produces red wine or another fruit wine, wherein fermentation is conducted solely on the juice of the fruit, separate from the fruit biomass. If the crushed grapes and juice are to be used in a red or “orange” style wine, they are processed in Step B1 106, which entails loading the crushed grapes and juice into the fermentation tank where yeast is added to initiate alcoholic fermentation. The winery may also produce a white wine or an on-fruit fermentation, so this embodiment is not restricted to a winery that only produces red wine. The winery may produce both red and white wine. This embodiment pertains to the process involving red wine production.

One embodiment of installing and using a system for the conversion of winery derivatives into bioactive products comprises the steps of:
a) a business delivers one or more bioreactors to a winery prior to crush;
b) winery staff transfers fruit biomass (marc) to one or more bioreactor(s);
c) winery staff rehydrates fruit biomass (marc) until berries swell, grinds the biomass into a meal, inoculates with microbials, and allows it to ferment at the winery facility;
d) winery staff transfers first-rack lees to the one or more bioreactors comprising fermenting meal;
e) winery staff emulsifies the first-rack lees and fermenting meal to generate puree, inoculates the puree and allows it to ferment at winery facility;
f) winery staff monitors the progress of fermenting puree and notifies the company when the fermentation bas completed; this could be supplemented by remote monitoring technology; and
g) the business picks up the one or more bioreactors and continues processing the fermented puree at the processing facility.
   Possible Variations on this General Process Include:

In some embodiments, it may be desirable to generate a “blank slate” effect in the microbial population of the fruit biomass, especially after the red wine fermentation process. This would provide for a more standardized fermentation process. One example comprises adding “killer yeast,” which have been modified to secrete an antibiotic against other yeast. Another example is sterilization via pasteurization, ozonation and or UV-radiation.

The business also remotely monitors the progress of the fermentations in the bioreactors

The Bioreactors are transferred to processing facility.

### Winery Producing White Wine

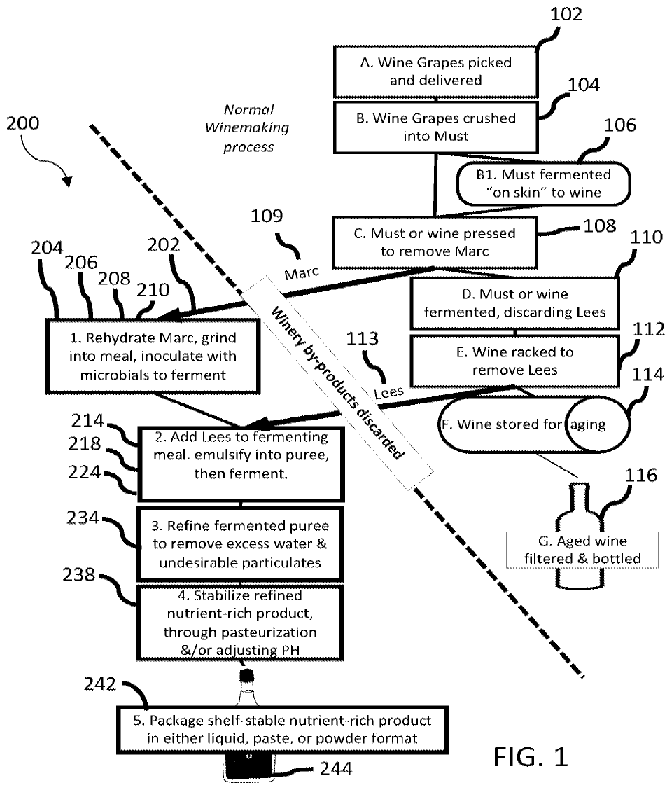

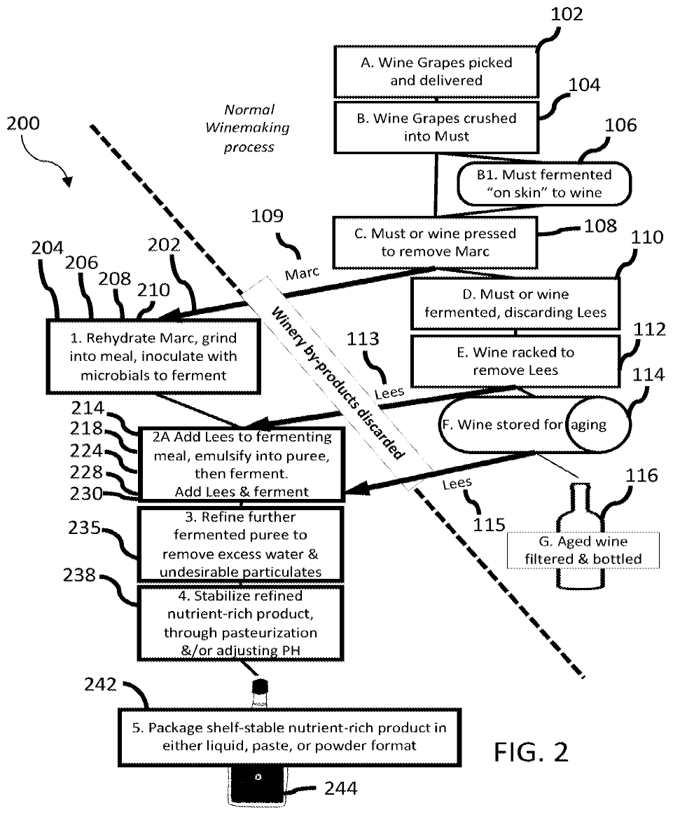

One embodiment of the system is deployed in a winery that produces white wine or another fruit wine, wherein fermentation is conducted solely on the juice of the fruit, separate from the fruit biomass. As described in FIGS. 1 and 2, if the crushed grapes and must are to be used in a white or rose style wine, they are immediately treated according to the process of Step C 108. The winery may also produce a red wine or an on-fruit fermentation, so this embodiment is not restricted to a winery that only produces white wine.

Ensuring that biomass does not begin wild ferment to ethanol as prohibited by certain religious dietary laws.

The Bioreactors are transferred to processing facility.

### Processing Facility

### Raw Nutrient-Rich Product Refinement Process

During Step 3 234, the fermented purée 225 or optionally, the further-fermented purée 232 is refined using filtration, homogenization, other techniques, or a combination of techniques in order to convert the fermented puree 225 or the further-fermented purée 232 into a refined nutrient-rich product 236. At this step, excess water is removed along with any undesirable particulates or bi-nutrient-rich products, such as sulfur, bentonite, etc. Appropriate adjustments can be made to generate a refined nutrient-rich product 236.

Some examples of steps that one may choose to employ include the following.
During Step 3 234 the fermented puree 225 or the further-fermented purée 232 will first be tested for bentonite, sulphur and other food contaminants (likely before removal from the winery).
In general, at this stage as described in more detail below during the work flow section, the bioreactor(s) 300 will be collected by the Business to continue processing within the business facility.
If contaminants are present, the fermented purée 225 or the further-fermented purée 232 will be likely be processed in a different manner that will be used only for livestock feed or nutrient extraction.
If sulphur is present, the fermented purée 225 or the further-fermented purée 232 is treated with a sulphur extractant (eg hydrogen peroxide) to remove the sulfur. (Sulfur in wines is mostly present as sulfite ion or as sulfurous acid. Atomic sulfur is almost undetectable. Sulfite in wines is either derived from additions to use as an antioxidant or from the metabolism by yeast of sulfur containing amino acids cysteine and methionine). The purée then would be homogenized, dewatered to a specific water %, and stored, usually by varietal.
The stored material could optionally be blended with other varietals (if necessary) to achieve a consistent flavour and nutrient profile. There may be an optional homogenization step after blending to stabilize to purée (keep the water from separating). This step also may involve dewatering.

Once the objectives for the chemical characterization of the nutrient-rich product have been met, the material is considered to be final nutrient-rich product 236.

### Step 4 Refined Nutrient-Rich Product Stabilization Process

During Step 4 238, the refined nutrient-rich product 236 is rendered shelf-stable through pasteurization or correction of ph level through further fermentation in order to convert the refined nutrient-rich product 236 into a stabilized nutrient-rich product 240. The desired qualities and characteristics for the end nutrient-rich product, will determine what criteria to look for at this stage of the process and will make the appropriate adjustments to generate an appropriate stabilized nutrient-rich product 240.

There are applications for both a pasteurized purée (with no bioactive materials) and a probiotic purée. High-pressure pasteurization technology will normally be used to create a pasteurized nutrient-rich product, although other current or future pasteurization techniques may be employed. The bioactive puree will be fermented to an approved pH level for sealed storage at room temperature, refrigerated temperature, and/or frozen.

### Step 5: Nutrient-Rich Product Packaging Process

During Step 5 242 the stabilized nutrient-rich product 240 is packaged into consumer, culinary and/or industrial vessels in order to convert the stabilized nutrient-rich product 240 into a packaged nutrient-rich product 244. It is then stored for shipment. The stabilized nutrient-rich product 240 could be in liquid, purée, paste or powder format based on the needs of the end-user. The qualities and characteristics for the packaged nutrient-rich product 244, will determine what criteria to apply at this stage of the process to make the appropriate adjustments to generate an appropriate product.

The stabilized nutrient-rich product 240 will be optimized for extrusion into scaled containers, which are specific to the industry and application using it. For example, the stabilized nutrient-rich product 240 may extruded into consumer-sized sealed jars or bottles to generate packaged nutrient-rich product 244 designed for home use. Alternatively, stabilized nutrient-rich product 240 may be extruded into 4 liter/1 gallon sealed containers to generate packaged nutrient-rich product 244 designed for culinary use. Alternatively, stabilized nutrient-rich product 240 may be extruded into sealed 20 liter/5 gallon pails, or 1000 liter Intermediate Bulk Containers to generate packaged nutrient-rich product 244 designed or industrial food processing or pharmaceutical use.

### The Nutrient-Rich Product

The nutrient-rich product can be used in food preparation, to:
a. reduce the amount of sodium in a food formula
b. Preserve dairy, meat, condiment and cereal nutrient-rich products
c. Enhance the flavour of fruits, vegetables, and spices within a food formula
d. Provide significant nutrient value to a food formula
e. Provide a source of yeast and other bacteria to cause the leavening of bread
f. Provide a source of bacillus to cause the fermentation of dairy nutrient-rich products
g. Provide a source of bacteria to cause the fermentation of plant-based proteins

The nutrient-rich product may also be used to provide a medium for extraction of nutrients for pharmaceutical use in addition to provide a medium for topical applications in cosmetics or skin therapy.

### A Business Method of Using the System

There are a number of different ways that the business method could be structured to appropriately integrate the system, and processes described herein in a fruit processing facility such as an orchard or a winery. The following non-limiting description provides one embodiment for how a business method of using the system could be structured.

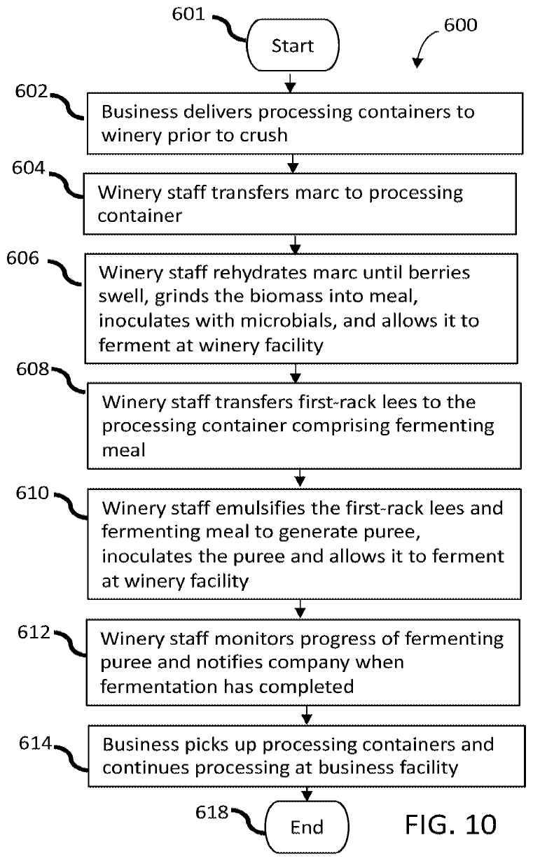

One embodiment as outlined in FIG. 10, entails the following steps. At step 602, a business delivers one or more transportable bioreactor(s) 300 to a winery prior to crush. At step 604, winery staff transfers marc to one or more transportable bioreactor(s) 300. At step 606, winery staff rehydrates marc until berries swell, grinds the biomass into a meal, inoculates with microbials, and allows it to ferment at the winery facility. At step 608 winery staff transfers first-rack lees to the one or more bioreactor(s) 300 comprising fermenting meal. At step 610 winery staff emulsifies the first-rack lees and fermenting meal to generate purée, inoculates the puree and allows it to ferment at winery facility. At step 612 winery staff monitors the progress of fermenting puree and notifies the company when the fermentation bas completed. At step 614, the business picks up the one or more transportable bioreactor(s) 300 and continues processing the fermented puree at the business facility.

In one embodiment, the business and the winery may choose to further process the fermented purée at the winery facility. In one embodiment, the business may choose to pick-up the transportable bioreactors prior to adding the first-rack lees 113, and conduct the further steps at the business facility. In this embodiment, the business may choose to collect the first-rack lees 113 from the winery when it is ready and add it to the fermenting meal 212 in the transportable bioreactor(s) 300 at the business facility. There are many different ways that this business relationship could be structured to optimize the resources of the business and the winery, such that these embodiments are considered to be non-limiting examples of how the workflow of the business relationship could be designed.

One embodiment as described in Example II, entails the business delivering transportable bioreactors to a winery prior to Step A 102 of the winemaking process, and retrieving them after fermentation has been completed and fermented purée 225 has been generated within transportable bioreactor(s) 300. The business benefits by having the initial steps of the process conducted on site at the Winery. This point saves the business from having to construct facilities on its location for Steps 1 and 2, and can focus the design of the Business facilities to processing the various nutrient-rich products.

The business could pay the winery for:
a) The amount of properly fermented purée potentially adjusted for:
   1. Level of solids with purée
   2. Type of varietal grapes used in the purée
   3. Whether the grapes are organic
   4. From a publicly recognized premium district, estate or vineyard.
   5. Distance from the processing center
b) Additional work or services independent of the amount of purée acquired, such as providing electrical power to the location or providing access to vineyard property during off bours.
c) Participating in nutrient-rich product development field testing.

In addition, the winery benefits by:
a) reduction of waste and costs associated therewith;
b) possibly the acquisition of Carbon Credits
c) an additional revenue stream;
d) incorporation of named winery purée into premium foods; and
e) eliminating methane emissions caused by disposal in buried landfills

The winery also benefits by diverting the substances from waste management and disposal processes to the conversion process, because these bioactive by-products are subject to numerous local health, environmental and worker safety regulations in the post-production treatment and disposal. The impact of these regulations on the winery are minimized.

Various components of the system described above may be packaged together in a kit for distribution, according to certain implementations. The kit may be assembled on site (e.g., the fruit processing facility or a franchisee? Or local operator).

### EXAMPLES

### Example 1: A Detailed Description how to Use the System in the Winemaking Industry

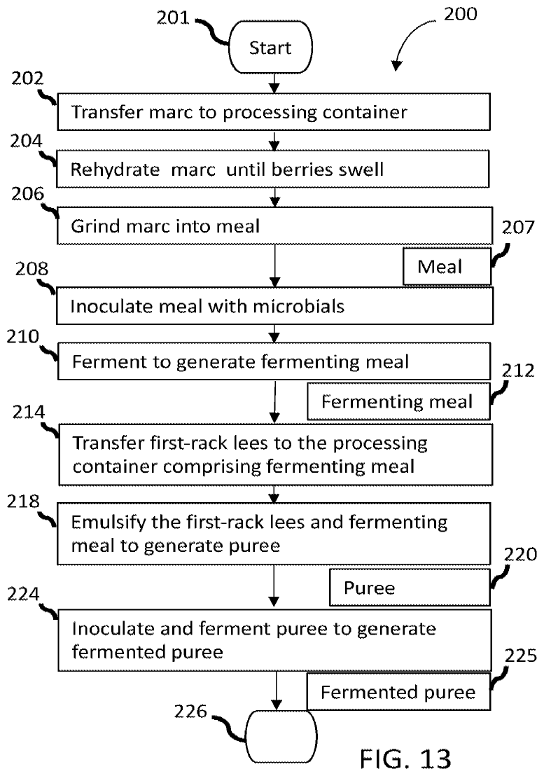

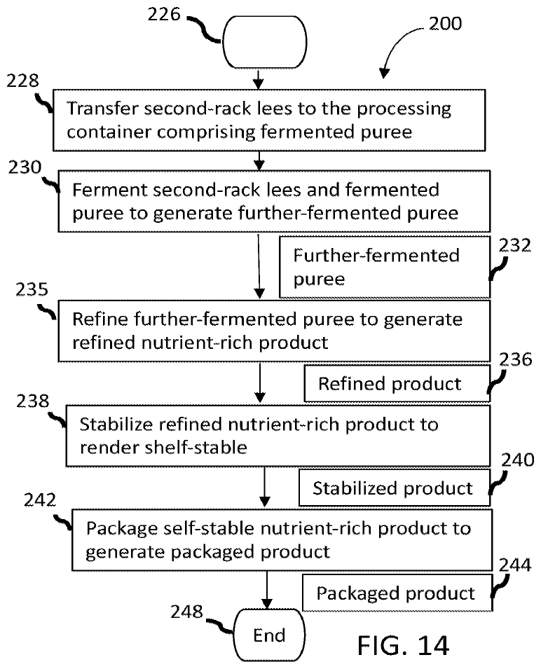

With reference to FIGS. 13, 14, 15 and 16, further details of these steps involved in the conversion of wine derivatives to nutritious products. This system, methods, processes and nutrient-rich products made thereby 200 could be adapted for wineries producing fruit wine. In one embodiment, the system 200 is integrated with a winery that produces fruit wine. One embodiment, the system 200 is integrated with a winery that produces both grape wine and fruit wine. In one embodiment, the system, methods, processes and nutrient-rich products made thereby 200 include the derivatives produced by both the grape winemaking process and the fruit winemaking process.

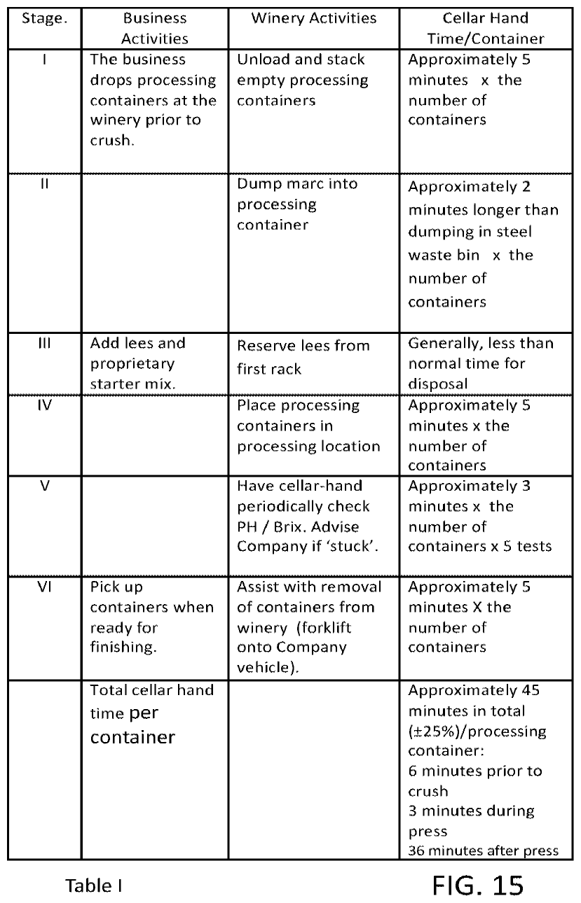

As illustrated by FIGS. 1 and 2, the process of derivative-conversion, begins by transferring mare 109 to one or more bioreactors 300 (illustrated in FIG. 3) of this system to be processed during Step 1 204, 206, 208, 210 (illustrated in FIGS. 13 and 15).

FIGS. 13, 14, 15 and 16 outline the steps described above, including the sub-steps constituting the steps, intermediaries and nutrient-rich products involved in embodiments of the system, methods processes, and nutrient-rich products made thereby. For example, Step 1 204, 206, 208, 210 comprises four sub-steps and various intermediates. The details for each of the steps and sub-steps are described herein.

### Step 1 Initial Acetic Acid Fermentation

As described in FIGS. 13 and 15, the marc 109 derived from the press is transferred to a bioreactor 300 of the system. Sub-step 202 is conducted by transferring the mare 109 from a collection vessel typically used in a winery to collect marc 109 from the press and pouring the contents into a bioreactor 300. The mare 109 is then rehydrated with an amount of water for a sufficient period of time to allow the berries to swell. In one embodiment, a bioreactor 300 is filled to 50-80% of its capacity, depending on the amount of mare available from the press. The addition space is reserved for lees and to provide sufficient air above the cap of the mixture to accelerate aerobic fermentation.

Once the berries have swollen, the biomass is ground into a meal 205. In one embodiment, the grinding process involves the use of a portable puréeing device that is inserted into the tank, which shreds the skins and pulp, and cuts up the seed, allowing the microbial “cocktail” to attack the grape skin particles, pulp, seed pulp and bruised husk. In one embodiment, a macerating pump, attached to the spigot that draws out the material, passing it though a grinder, and then pumping it back into the top of the container.

The meal 205 is then inoculated with a microbial culture that may comprise bacteria (e.g., acetic acid bacteria), enzymes and/or yeast to cause fermentation and dissolution of solid particles. Optionally, sugar or other natural sweetener may be added to accelerate the fermentation. The inoculated meal then is aeriated at a level that introduces enough oxygen to allow aerobic fermentation to dominate all bioactivity. The inoculated meal 205 is then allowed to rest and begin fermentation and thereby becomes fermenting meal 212. The bioreactors are checked periodically to monitor the progression of fermentation as assessed by the pH, the Brix level, optionally the temperature. The requirements of a specific of fermentation will determine which factors would be most relevant to the type of nutrient-rich product one is seeking to generate.

This process can generally last for 2 to 6 weeks. As this system is integrated with the general winemaking process, as depicted in FIGS. 1 and 2, the time-period for this process can depend upon the availability of the lees generated by the winemaking process.

### Step 2: Lee-Addition and Continued Fermentation

As described above, within the General Overview section, Step E 112 of the process of conventional winemaking, wherein either the must or pressed wine (red) is racked, generates first-rack lees 113. The first-rack lees 113 is typically considered food waste and immediately removed from the “food preparation” area. Wineries will either syphon the wine from the top of the fermentation tank until the wine becomes cloudy, or will drain the wine from the bottom of the tank using a filtration system to remove lees particles.

First-rack 113 lees can be collected in food safe containers and then poured over the fermenting meal 212 in a bioreactor 300 during sub-step 214. During sub-step 218, the first-rack lees 113 and the fermenting meal 212 are emulsified to generate a purée 220. The purée is then fermented in sub-step 224, during which the pH of the purée will be expected to drop to below 3.9 pH.

### Optional Inoculation of the Purée

One objective for this fermentation process is to establish how “sour” the ultimate fermented purée 225 should be allowed to become. Care must be provided regarding the levels of acetic acid present during the fermenting process. Ideally the optimal pH can be attained with the lactic acid remaining in the first-rack lees 113.

As described in the General Overview section, step D 110 of the general red winemaking process, involves inoculating the press wine produced from Step C 108, with specific strains of bacteria (lactobacter) to initiate malo-lactic fermentation to convert malic acid to lactic acid to soften the taste of the wine. If there isn't sufficient lactic acid remaining in the first-rack lees 113 after the malo-lactic fermentation, the purée 220 in bioreactor 300 is inoculated with a microbial solution comprising acetic acid bacteria during sub-step 224 and allowed to ferment for approximately 60-90 days.

The fermentation process is monitored by periodically checking the PH/Brix ratio to maintain the pH of the purée 220 below 4.5 pH. This stage of the process is considered finished when the pH drops to an appropriate level, likely around 3.9 or possibly less, and the purée 220 is considered completely converted to fermented purée 225. The bioreactors are checked periodically to monitor the progression of fermentation as assessed by the pH, the Brix level, optionally the temperature. The requirements of a specific fermentation determine which factors would be most relevant to the type of nutrient-rich product one is seeking to generate.

### Optional Storage-Lee-Addition Process

In one embodiment, illustrated in FIG. 5, an additional lee transfer step is included in the derivative-conversion process. During Step F 114 of the conventional winemaking process, the wine is stored for aging, which generates second-rack lees 115. This embodiment entails collecting and transferring the second-rack lees 115 to bioreactor 300 during sub-step 228.

This optional step is performed in a manner similar to the collection and transfer of the first-rack lees 113, described above. In this collection and transfer step, however, care must be taken to ensure that the winery did not employ any filtration catalysts, such as bentonite (clay), egg whites (non-vegan), that would contaminate the final nutrient-rich product. If these other substances are present, appropriate applications would will determine the final nutrient-rich product that will eventually be produced and what should be avoided.

During sub-step 230, the bioreactors are checked periodically to monitor the progression of fermentation as assessed by the pH, the Brix level, optionally the temperature, etc. The requirements of a specific fermentation will determine which factors would be most relevant to the type of nutrient-rich product they are seeking to generate. Once the desired factors are present within the biomass, the further fermented puree 232 will be refined according to Step 3 324.

### Example II

With reference to Table 1 presented in FIG. 17, this example describes one non-limiting manner in which the system, methods, processes and nutrient-rich products 200 made thereby can be incorporated into a winery producing grape wine. The Business is used to denote the business practicing the business methods described herein. The Winery is used to denote the wine production business within which this system, methods, and processes 200 is integrated.

### Phase I. At the Winery

Table 1 shows the main stages in column 1, wherein the employees of the Winery (referred to herein as “cellar-hand”) are instructed to perform task(s) involved in the processes of this system, methods, processes 200. The Business activities are presented in column 2, the Winery's activities in column 3, and estimated cellar-hand time per container in column 4. It is estimated that cellar-hand activities will be less than one hour per container over the ≈140 days that the containers are on site at the Winery.

### Stage 1: Pre-Crush

The Business drops bioreactors 300 at the winery prior to crush. The bioreactor 300 may already have an initial microbial cocktail encased in the interior compartment of the bioreactor 300, which will become activated once water is added to the bioreactor 300.

The numbers of bioreactors 300 could be based on the following ratio: anticipated red grape tonnage×25% (average percentage of mare 109). For example, if a winery accepts 100 tons of red grapes, the Business could deliver 25 bioreactors 300. Empty bioreactors 300 could be stacked 2-3 high in a place where they least impact crush activities. One non-limiting example of where empty bioreactors 300 could be stored on the grounds of the Winery is illustrated in FIG. 1.

### Stage II During Press (Crush): Transfer Mare to Bioreactor

Pursuant to sub-step 202, the cellar-hand is instructed to collect, transfer and deliver marc 109 generated during Step B 104 (crush), using a collection bin that is normally used to collect mare 109 from the press. Rather than discarding the mare 109 as per the usual winemaking process, wherein the marc 109 is usually dumped into a steel disposal bin, the cellar-hand is instructed to place the mare 109 into bioreactors 300. The cellar-hand is instructed to fill the bioreactor 300 until the marc 109 fills up to the 600-ltr level of the bioreactor 300, and then instructed to add sufficient water to saturate and cover the mare 109, allowing the berries to swell as per sub-step 204, and to close and secure the lid 334. This step generally requires less than 6 minutes to for the cellar-hand to perform, which is slightly longer than if they were to dump the mare 109 into a disposal bin as per the traditional process. After the berries have swelled, the hydrated marc may be optionally macerated using a motorized high-sheer mixer that would break the seeds and skin. This accelerates the fermentation process and seed decomposition. This step may be delayed until after the lees are added.

If necessary, the cellar-hand can move and/or restack the bioreactors 300 to minimize the impact of the presence of bioreactors 300 on space requirements of the crush activities. One example of where bioreactors 300 can be placed is illustrated in FIGS. 1, 9 and 10. The biomass in the bioreactors 300 is allowed to rest and begin fermentation from natural bacteria and/or added microbials, while waiting for first-rack lees 113.

### Stage III: Transfer First Rack Lees to Bioreactor

When Step E of the winemaking process is completed, the cellar-hand is instructed to collect and transfer the first-rack lees 113 to the bioreactor(s) 300, pursuant to sub-step 213 of the process. The time estimate for performing sub-step 213 is approximately 1-2 minutes×19 ltr (five gallon) pails per container, which is generally takes less amount of time than the usual time required to dispose of first-rack lees 113. Once the lees are mixed into the marc, the combined material can be macerated using a high-sheer mixer, either for the first time if this did not happen in Stage II, or as secondary maceration to further break down the purée.

### Stage IV: Prior to Microbial Fermentation

After sub-step 224 has been performed, the cellar-hand is instructed to place bioreactor(s) 300 together in vineyard row(s) reasonably close to an electric power source. The cellar-hand is instructed to place bioreactor(s) 300 where it would be convenient, and where CO2 sequestering could be maximized. The more containers that are lined up, the better as they will maintain internal heat. A protective cover could then be placed over a series of the containers. The cover will be tamper-resistant, will capture solar heat, and will retain heat from both the fermentation process and solar capture. They can also be used as a windbreak if desired. One example of where bioreactors 300 can be placed is illustrated in FIGS. 1, 9 and 10. The cellar-hand is instructed to let bioreactor(s) 300 ferment for 60-90 days.

### Stage V: During Microbial Fermentation

During this fermentation period, the cellar-hand is instructed to periodically check the PH/Brix measurements of the fermenting puree in the bioreactor(s) 300. Should the purée in a specific container show signs of stabilization (pH and BRIX levels stay constant for an extended period), the winery would advise the Business of the situation.

### Stage VI Post Microbial Fermentation

When the fermentation has been deemed to be finished, the Business picks up the bioreactor(s) 300 for processing at the Business facility. The cellar-hand will generally assist in this process, for example, using winery forklifts to transfer the bioreactor(s) 300 to the Business truck. This is likely 90-120 day after marc 109 is pressed. For example, if Merlot grapes were pressed on November 1, the container could be ready between February 1 and March 1. If Cabernet Sauvignon grapes were pressed December 1, the bioreactor 300 would be ready for pick-up by the Business approximately March 1-April 1. The bioreactor 300 would be ready for shipment from the winery to the Business at this time.

### Phase II: At the Business Facility

The fermented purée 225 is processed at the Business facility using Business staff. In brief, the steps generally include that Business staff:
transfers the bioreactors to Business facility;
empties the contents of bioreactor(s) 300 into a blending tank with other varietals to achieve a consistent blend;
emulsifies and homogenize the fermented purée 225 to mitigate and/or remove oversized grape seed husk;
packages stabilized nutrient-rich product 240 new bioreactor(s) 300; and
ship packaged nutrient-rich product 244 to one or more food processor(s)

While the foregoing description and FIGS. represent the certain implementations of the present invention, it will be understood that various additions, modifications, combinations and/or substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof. The invention may be used with many modifications of structure, arrangement, proportions, materials, and components and otherwise, used in the practice of the invention, which are particularly adapted to specific environments and operative requirements without departing from the principles of the present invention. In addition, features described herein may be used singularly or in combination with other features. The presently disclosed implementations are, therefore, to be considered in all respects as illustrative and not restrictive, the scope of the invention being indicated by the appended claims and not limited to the foregoing description.

It will be appreciated by those skilled in the art that changes could be made to the implementations described above without departing from the broad inventive concept thereof. It is understood, therefore, that this invention is not limited to the particular implementations disclosed, but it is intended to cover modifications within the spirit and scope of the present invention, as defined by the following claims.

1. A system comprising a transportable bioreactor configured for: a) transport via road, rail and/or a body of water, compliant with regulatory requirements; b) fermentation compliant with sanitation regulations; c) collecting fruit biomass at a fruit processing facility, and d) for initiating fermentation at a selected time starting after the fruit biomass is collected in the container, comprising:
a. a transportable bioreactor comprising a food-grade container configured for fermentation; accessibility for filling, emptying and monitoring and cleaning, further comprising:
i. means to exclude external contamination; and
ii. means to prevent spillage during transport.

2. The system of claim 1, further comprising means for introducing a microbial formulation to initiate fermentation of the biomass in the transportable bioreactor at a selected time.

3. The system of claim 1, wherein the fruit processing facility is a winery.

4. The system of claim 1, further comprising system monitoring means.

5. A process for collecting fruit biomass at a fruit processing facility within a transportable aerobic bioreactor, comprising the steps of:
providing a transportable aerobic bioreactor at a fruit processing facility;
transferring fruit biomass into the transportable aerobic bioreactor at the fruit processing facility;
inoculating the fruit biomass within the bioreactor with a microbial formulation;
aerobically fermenting the inoculated biomass in the bioreactor until the acidity is at or below pH 4.0;
transporting the bioreactor at a selected time after aforesaid step of transferring fruit biomass, from the fruit processing facility to a processing facility for further processing into a product.

6. The process of claim 5, wherein the microbial formulation comprises Acetobacter, and/or Gluconobacter.

7. The process of claim 5, wherein the microbial formulation comprises one or more strains of yeast that can ferment under aerobic, microaerophilic and/or anaerobic conditions.

8. The process of claim 5, wherein enzymes are added to the bioreactor.

9. The product produced by the process of claim 5.

10. The process of claim 5, wherein the fruit processing facility is a winery.

11. The process of claim 5, wherein the fruit biomass comprises marc with berries and the process additionally incorporates lees derived from a winemaking process, further comprising the steps of:
a. grinding the hydrated fruit biomass comprising marc in the bioreactor to generate meal;
b. inoculating the meal in the bioreactor with a microbial formulation;
c. fermenting the inoculated meal in the bioreactor to generate a fermenting meal.

12. The process according to claim 11, additionally, after step a, hydrating said fruit biomass comprising marc in the bioreactor until berries swell.

13. The process according to claim 11, additionally
introducing lees into the bioreactor;
emulsifying the lees and fermenting meal in the bioreactor to generate a puree;
transporting the bioreactor to a processing facility where the puree is refined to generate a nutrient-rich product;
whereby methane emissions that would otherwise be caused by disposal of fruit biomass in landfills are significantly reduced or eliminated.

14. The process of claim 13, wherein the lees are first-rack lees.

15. The process of claim 13, further comprising the steps of inoculating the puree in the bioreactor and fermenting the inoculated puree to generate a fermented puree in the bioreactor.

16. The process of claim 15, further comprising the step of refining the fermented puree to generate a refined nutrient-rich product.

17. The process of claim 16, further comprising the steps of stabilizing the refined nutrient-rich product to generate a stabilized nutrient-rich product, and then packaging the stabilized nutrient-rich product.

18. The process of claim 13, wherein the microbial formulation comprises acetic acid bacteria and/or fungus inoculant.

19. The process of claim 18, wherein the microbial formulation comprises Acetobacter, and/or Gluconobacter.

20. The process of claim 11, wherein enzyme is added to the fermenting meal.
